(19) United States
(12) Patent Application Publication
(10) Pub. No.: US 2026/0269023 A1
(43) Pub. Date: Sep. 10, 2026

(54) ESTIMATION MODEL GENERATION METHOD/GENERATION DEVICE FOR ESTIMATING REACTIVE CONDITION, REACTIVE CONDITION PROVIDING METHOD/PROVIDING DEVICE, AND PROGRAM

(71) Applicant: NIPPON SODA CO., LTD., Tokyo (JP)

(72) Inventor: Katsuma TAKEHISA, Takaoka-shi (JP)

(73) Assignee: NIPPON SODA CO., LTD., Tokyo (JP)

(21) Appl. No.: 18/867,915

(22) PCT Filed: Jun. 19, 2023
(86) PCT No.: PCT/JP2023/022524

§ 371 Date: Nov. 21, 2024

(30) Foreign Application Priority Data
Jul. 6, 2022 (JP) 2022-109322

(51) Int. Cl. G16C 20/70; G16C 20/10

(52) U.S. Cl. CPC G16C 20/70; G16C 20/10

(57) ABSTRACT

In the present invention, under a condition that acquired information on a plurality of chemical substances as reaction objects and a product is similar to information on a plurality of chemical substances and a product set to a reactive condition, a plurality of the acquired reactive conditions when a plurality of chemical substances as reaction objects are reacted are set from reaction items set to the reactive condition. Furthermore, using an estimation model in which machine learning has been executed with chemical structure information and physical property information of a plurality of chemical substances reacted in the past, reactive conditions in the reactions, chemical structure information and physical property information of produced products, and yields when the reactions are performed under the reactive conditions as training data, the yield is estimated for each of the plurality of reactive conditions, and the reactive condition under which the yield among the estimated yields meets a predetermined condition is displayed (output).

### TECHNICAL FIELD

The present invention relates to a generation method of an estimation model for estimating a reactive condition under which a yield meets a predetermined condition from information on a plurality of chemical substances and information on a product, and a generation device of the estimation model, a providing method of the reactive condition and a providing device of the reactive condition, and a program.

### BACKGROUND ART

Conventionally, chemists have generated desired products by chemically reacting a plurality of chemical substances. Additionally, for efficiently generating the desired products, the chemists repeatedly perform chemical experiments while changing a reactive condition for each experiment based on their own empirical rule and the like, thus searching the reactive conditions under which the yield meets a predetermined condition (for example, a reactive condition providing a higher yield, and the like).

However, for searching the reactive conditions under which the yield meets a predetermined condition, it is necessary that the chemist determines the reactive condition by oneself by trial and error and repeatedly performs the chemical experiment. Therefore, enormous man-hours for operation are required, and there has been a problem of improving the operation efficiency. To deal with the problem, there has been known a flow synthesis technique enabling automatically setting a reactive condition appropriate for a synthesis reaction by reflecting an analysis result by an artificial intelligence (for example, patent literature 1).

Patent Literature 1 discloses a flow synthesis device that automatically sets a reactive condition having at least one of a reaction temperature, a reaction time, a reagent type, a reagent amount, and a solvent as a component. The flow synthesis device is controlled by an information processing device that analyzes synthesis reaction information included as a database with an artificial intelligence having a structural fingerprint and a reactive condition as explanatory variables.

### CITATION LIST

### Patent Literature

Patent Literature 1: Japanese Unexamined Patent Application Publication No. 2019-185506

### SUMMARY OF INVENTION

### Object to be Solved by the Invention

However, in the technique disclosed in Patent Literature 1, a vector combining only structural fingerprint vector information and reactive condition vector information is used as an explanatory variable, and the machine learning is executed based thereon (that is, physical property information (information indicative of physical and/or chemical property of a chemical substance) is at least not considered as the explanatory variable). Therefore, the t accuracy of the generated prediction model is not sufficient, consequently, the yield possibly does not reach a desired level under the set reactive condition, and the improvement of such a situation has been desired.

The present invention has been made in consideration of the above-described conventional problem, and has an object to reduce a man-hour for operation in setting a reactive condition under which a yield meets a predetermined condition.

### Means to Solve the Object

That is, an estimation model generation method of the present invention includes: an acquiring step of acquiring information on a plurality of chemical substances reacted in a past, reactive conditions in the reactions, information on a product produced by reacting the plurality of chemical substances, and yields when the reactions are performed under the reactive conditions; a first generating step of generating training data from chemical structure information and physical property information derived for each piece of the information on the plurality of chemical substances reacted in the past, the reactive conditions, chemical structure information and physical property information derived for the information on the product, and the yields; and a second generating step of generating an estimation model having information on a plurality of chemical substances to be reacted and information on a product to be produced as input values and a reactive condition under which a yield of the product to be produced meets a predetermined condition as an output value by executing machine learning using the training data.

A reactive condition providing device of the present invention includes: acquiring unit that acquires information on a plurality of chemical substances as reaction objects and information on a product to be produced; storing unit that stores one or more reactive conditions; setting unit that sets a plurality of reactive conditions for reacting the plurality of chemical substances as the reaction objects from reaction items set to the one or more reactive conditions when a similarity between the information on the plurality of chemical substances as the reaction objects and the information on the product to be produced and information on a plurality of chemical substances and information on a product set to the reactive conditions is equal to or more than a predetermined threshold; estimating unit that estimates a yield for each of the plurality of reactive conditions from chemical structure information and physical property information derived for each piece of the information on the plurality of chemical substances as the reaction objects, chemical structure information and physical property information derived for the information on the product to be produced, and the plurality of set reactive conditions, by using an estimation model in which machine learning has been executed with chemical structure information and physical property information derived for each piece of information on a plurality of chemical substances reacted in a past, reactive conditions in the reactions, chemical structure information and physical property information derived for information on products produced in the reactions of the plurality of chemical substances, and yields when the reactions are performed under the reactive conditions as training data; and display control unit that causes a display device to display the reactive condition under which the yield among the yields estimated for the respective reactive conditions by the estimating unit meets a predetermined condition.

### Effect of the Invention

According to the present invention, the man-hour for operation can be reduced in setting the reactive condition under which the yield meets the predetermined condition.

### BRIEF DESCRIPTION OF DRAWINGS

FIG. 1A is a drawing illustrating a model generation device.

FIG. 1B is a drawing illustrating a reactive condition providing device.

FIG. 2 is a block diagram illustrating a functional configuration of the model generation device.

FIG. 3 is a drawing illustrating chemical structure information of benzene.

FIG. 4A is a block diagram illustrating functional configurations of a first feature quantity reduction unit of a feature quantity selection unit.

FIG. 4B is a block diagram illustrating functional configurations a feature quantity selection model generation unit of a feature quantity selection unit.

FIG. 5A is a block diagram illustrating functional configurations of a multi-estimation-model generation unit of an estimation model generation unit.

FIG. 5B is a block diagram illustrating functional configurations of an estimation model mixing unit of an estimation model generation unit.

FIG. 6 is a flowchart illustrating a procedure for a process of generating an estimation model in the model generation device.

FIG. 7 is a flowchart illustrating a procedure for a process of deriving a molecular descriptor in a molecular descriptor derivation unit.

FIG. 8 shows drawings illustrating a generation process of a data set (training data).

FIG. 9A is a drawing illustrating missing value processing in a missing value processing unit.

FIG. 9B is a drawing illustrating missing value processing in a missing value processing unit.

FIG. 9C is a drawing illustrating missing value processing in a missing value processing unit.

FIG. 9D is a drawing illustrating missing value processing in a missing value processing unit.

FIG. 10 is a drawing illustrating a process in a category value conversion unit.

FIG. 11 is a flowchart illustrating a procedure for a process of selecting a feature quantity (feature quantity group) in the feature quantity selection unit.

FIG. 12 is a flowchart illustrating a procedure for a process of reducing (deleting) the feature quantity in the first feature quantity reduction unit.

FIG. 13A is a drawing illustrating a correlation coefficient and a process of reducing the feature quantity according to the correlation coefficient.

FIG. 13B is a drawing illustrating a correlation coefficient and a process of reducing the feature quantity according to the correlation coefficient.

FIG. 14 is a flowchart illustrating a procedure for a process of generating a feature quantity selection model in the feature quantity selection model generation unit.

FIG. 15 is a drawing illustrating a decision tree model.

FIGS. 16A-16C are drawings illustrating a process of reducing (deleting) the feature quantity in a second feature quantity reduction unit.

FIGS. 16D-16F are drawings illustrating a process of reducing (deleting) the feature quantity in a second feature quantity reduction unit.

FIG. 17 is a flowchart illustrating a procedure for a process of generating an estimation model in the estimation model generation unit.

FIG. 18 is a flowchart illustrating a procedure for a process of generating a plurality of estimation models in the multi-estimation-model generation unit.

FIG. 19 is a flowchart illustrating a procedure for a process of generating one estimation model among a plurality of estimation models in the multi-estimation-model generation unit.

FIG. 20 is a flowchart illustrating a procedure for a process of mixing a plurality of estimation models.

FIG. 21 is a flowchart for describing process contents (data flow) in the estimation model generation unit.

FIG. 22 is a block diagram illustrating a functional configuration of a reactive condition providing device.

FIG. 23 is a flowchart illustrating a procedure for a process of providing a reactive condition in the reactive condition providing device.

FIG. 24A is a drawing showing the information on a plurality of chemical substances and the information on a product, the information on a plurality of chemical substances reacted in the experiments performed in the past, the information on products produced as the result of reacting the plurality of chemical substances, and the information on the reactive conditions when the plurality of chemical substances is reacted.

FIG. 24B is a drawing showing the information on a plurality of chemical substances and the information on a product converted into the chemical structure information, the information on a plurality of chemical substances, the information on products produced as the result of reacting the plurality of chemical substances converted into the chemical structure information, and the information on the reactive conditions when the plurality of chemical substances is reacted.

FIG. 24C is a drawing for describing a similarity calculation process in the reactive condition providing device.

FIG. 24D is a drawing for describing a reactive condition selecting process.

FIG. 24E is a drawing for describing a reactive condition selection process.

FIGS. 25A-25C are drawings for describing process from S3-8 to S3-12 of the flowchart (FIG. 23) illustrating a procedure for a process of providing a reactive condition in the reactive condition providing device. FIG. 25A is a drawing for describing a reaction simulation executing process in the reactive condition providing device. FIG. 25B is a drawing for describing a rank assigning process. FIG. 25C is a drawing for describing a process of determining whether a reactive condition meeting a predetermined condition is present or not, and a selecting process of the reactive condition meeting a predetermined condition.

FIG. 26 is a drawing for describing the reaction simulation executing process.

FIGS. 27A-27E are drawings illustrating a function between the reactive condition and the yield, and a process of Bayesian optimization. FIG. 27A is a drawing illustrating a function between the reactive condition and the yield and a process of Bayesian optimization. FIG. 27B is a drawing illustrating a function between the reactive condition and the yield and a process of Bayesian optimization. FIG. 27C is a drawing illustrating a function between the reactive condition and the yield and a process of Bayesian optimization. FIG. 27D is a drawing illustrating a function between the reactive condition and the yield and a process of Bayesian optimization. FIG. 27E is a drawing illustrating a function between the reactive condition and the yield and a process of Bayesian optimization.

FIG. 28 is a drawing illustrating a normal distribution.

FIG. 29 is a flowchart illustrating a procedure for a process of identifying the reactive condition by executing the Bayesian optimization while Constant Liar Approach is applied.

FIG. 30 is a drawing illustrating a sample screen of the reactive conditions displayed on a display device.

FIG. 31 is a block diagram illustrating a hardware configuration of the model generation device.

### MODE OF CARRYING OUT THE INVENTION

The following describes embodiments of the present invention with reference to the drawings. The embodiments below do not limit the present invention, and all the combinations of the features described in the embodiments are not necessarily required for the solution of the present invention. Additionally, various configurations within a range not departing from the gist of the present invention are included in the present invention, and the following embodiments can be partially combined as necessary.

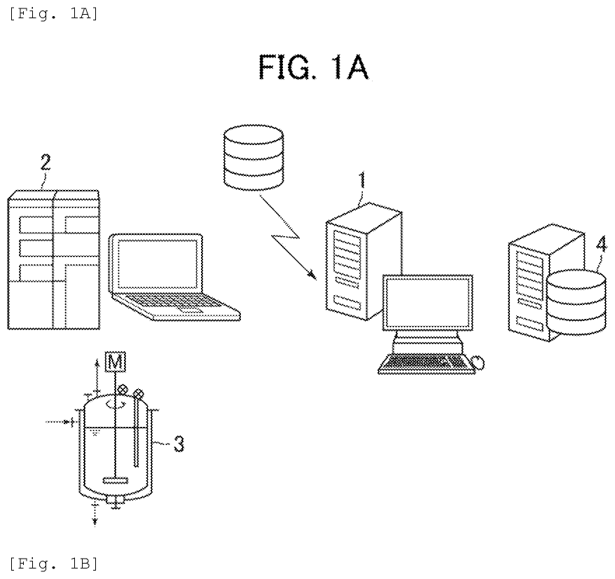

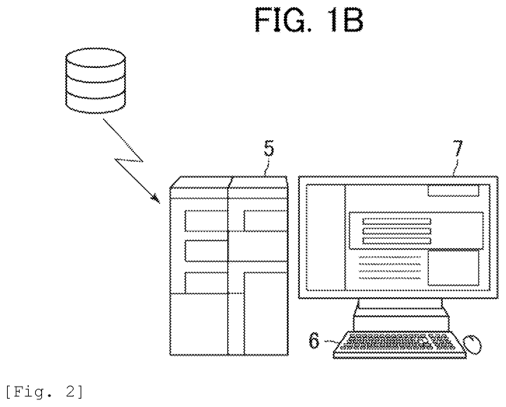

FIG. 1A is a diagram illustrating a model generation device and FIG. 1B is a diagram illustrating a reactive condition providing device according to this embodiment. A model generation device 1 illustrated in FIG. 1A generates training data from information on a plurality of chemical substances, reactive conditions when the plurality of chemical substances are reacted, information on a product produced by reacting the plurality of chemical substances, and yields of the product when the reactions are performed under the reactive conditions, which have been acquired as input data. The model generation device 1 executes machine learning using the generated training data to generate an estimation model (which is a regression model) having a reactive condition under which a yield of the product to be produced meets a predetermined condition as an output value. When a plurality of chemical substances is reacted, usually, a plurality of products is produced. Therefore, here, among the plurality of products, information on a product planned to be produced or information on a product that can be beneficial in generating the estimation model is input to the model generation device 1 as the information on the product produced by reacting a plurality of chemical substances.

While the information on a plurality of chemical substances, the reactive condition, the information on the product, and the yield can be input to the model generation device 1 using a predetermined input device, an analysis device 2 can be connected to the input side of the model generation device 1 as illustrated in FIG. 1A, and information on chemical substances and a reactive condition, and a product produced in a reactor 3 and a yield of the product can be directly input to the model generation device 1 from the analysis device 2 (more accurately, information processing device attached to the analysis device 2). An external storage device 4 can be connected to the output side of the model generation device 1, and a generated estimation model can be stored in the external storage device 4.

A reactive condition providing device 5 illustrated in FIG. 1B is a device that estimates a reactive condition in producing a product to be produced using chemical substances, and automatically provides the estimated reactive condition. The reactive condition providing device 5 uses an estimation model (regression model) generated by the model generation device 1 (FIG. 1A), and estimates a yield for each of a plurality of reactive conditions from: chemical structure information and physical property information derived from the information on a plurality of chemical substances acquired as input data from a predetermined operation device 6; chemical structure information and physical property information derived from the information on products produced by reacting the plurality of chemical substances; and the plurality of reactive conditions when the plurality of chemical substances are reacted that are set from one or more reaction items stored in a storage device (storage unit), based on chemical structure information and physical property information derived from the information on the plurality of chemical substances acquired as input data and chemical structure information and physical property information derived from the information on the products. The reactive condition providing device 5 outputs (displays) the reactive condition under which a yield among the yields estimated for the respective reactive conditions meets a predetermined condition to an external display device 7. The following sequentially describes embodiments of each of the model generation device 1 and the reactive condition providing device 5.

### (Model Generation (Model Generation Device))

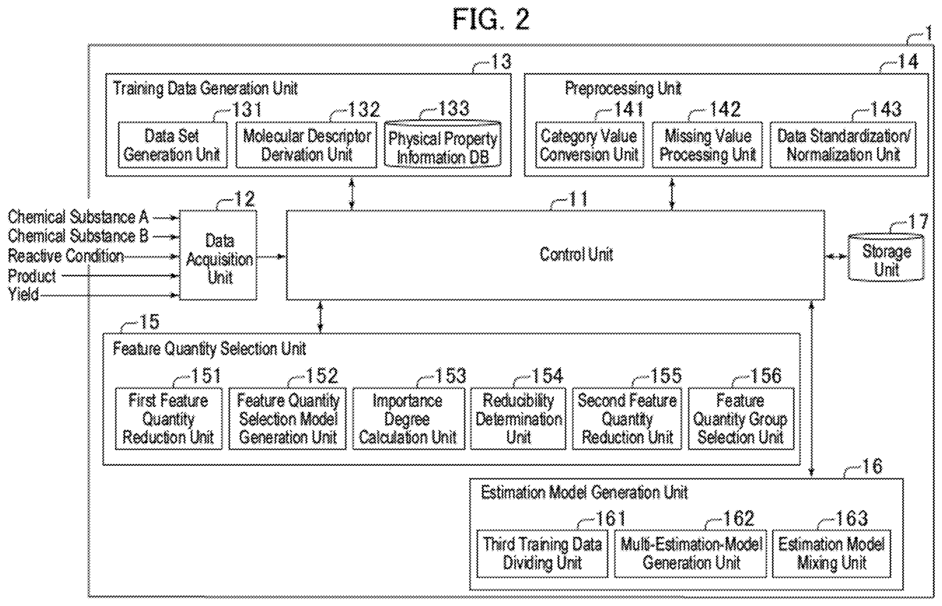

FIG. 2 is a block diagram illustrating a functional configuration of the model generation device 1 according to the embodiment. As illustrated in FIG. 2, the model generation device 1 includes, as the functions, mainly, a control unit 11, a data acquisition unit 12, a training data generation unit 13, a preprocessing unit 14, a feature quantity selection unit 15, an estimation model generation unit 16, and a storage unit 17.

The control unit 11 is a function block that controls processes of the respective function blocks. The data acquisition unit 12 acquires information on a plurality of chemical substances, a reactive condition, information on a product, and a yield. The various kinds of data (information) acquired by the data acquisition unit 12 is temporarily stored in the storage unit 17 by the control unit 11. Note that the information on the chemical substance and the information on the product can be described with the notation such as SMILES notation, MOL file, and SDF file, and in this embodiment, information described with SMILES notation is acquired. SMILES notation is a notation method for expressing a chemical structural formula by converting it into a letter string, and for example, benzoic acid can be notated as “OC(C₁═CC═CC═C₁)═O.” Additionally, here, as the information on a plurality of chemical substances, information on two chemical substances, information on a chemical substance A and information on a chemical substance B, is used in the explanation.

As the reactive conditions, a solvent, a temperature, a concentration, a drip rate, a drip method, a stirrer, and the like can be set, and among the reactive conditions, the reactive conditions (for example, temperature or the like) other than the reactive condition relating to a part of compounds are defined as control conditions. The yield is indicated as a ratio between a theoretical yield that can be theoretically acquired and the actually acquired amount when a product is to be obtained from a predetermined chemical substance.

The training data generation unit 13 includes a data set generation unit 131, a molecular descriptor derivation unit 132, a physical property information database (in the drawing, physical property information DB) 133, and generates training data from the various kinds of data acquired by the data acquisition unit 12. The data set generation unit 131 generates the training data as a predetermined data set based on (1) a molecular descriptor for the information on the chemical substance derived by the molecular descriptor derivation unit 132, (2) a molecular descriptor for the information on the product derived by the molecular descriptor derivation unit 132, (3) the reactive condition, and (4) the yield.

The molecular descriptor derivation unit 132 derives the molecular descriptors from the chemical structure information and the physical property information indicated as indices (numerical values) in determining the molecular structure. In the derivation of the molecular descriptor, in this embodiment, while Morgan method is used in the explanation, Mordred method, RDkit descriptor method, or the like can be used. When Morgan method is used, the chemical structure information is calculated, and the physical property information is acquired from a predetermined database, thereby deriving the molecular descriptor.

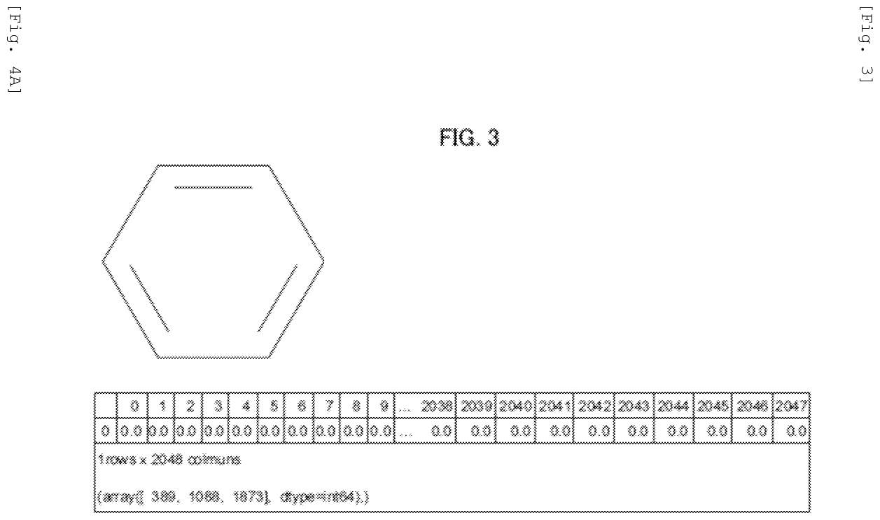

For the chemical structure information, for example, in the case of benzene, by converting partial structures into “0” and “1,” the calculation can be made as illustrated in FIG. 3. Specifically, for benzene, among the sequences from the sequence [0] to the sequence [2047], “1” is stored for the sequence [389], the sequence [1088], and the sequence (that is, partial structures are included), and “0” is stored for the other sequences. That is, the chemical structure information can be calculated such that “1” is stored for the sequence [389], the sequence [1088], and the sequence.

The molecular descriptor derivation unit 132 acquires physical property information from the physical property information database 133 that stores information (physical property information) indicative of physical and/or chemical properties of chemical substances. For example, for the solvent, a density, a refractive index, a permittivity, a dipole moment, a melting point, a boiling point, a viscosity, and the like can be used as the physical property information.

The physical property information includes information that cannot be estimated from only the chemical structure information. For example, even when the chemical substance is same (even when the chemical structure is same), when a crystal polymorphism is present, the melting point or the solubility has a different physical property value, and therefore, the yield of the chemical reaction is significantly affected in some cases. Therefore, in the generation of the estimation model, by taking the physical property information into consideration as well (by mixing the input from the physical property value information in a publicly known physical property database or input of an actual physical property measurement value), the accuracy improvement of the estimation model can be expected.

The molecular descriptors derived by the molecular descriptor derivation unit 132 (that is, the calculated chemical structure information and the acquired physical property information) are added to the data set as data (components) by the data set generation unit 131. Thus, the data set generation unit 131 adds the molecular descriptor to the information on the chemical substance as a component of the data set, similarly, adds the molecular descriptor to the information on the product as a component of the data set, and furthermore, adds the reactive condition and the yield acquired by the data acquisition unit 12, thereby generating the training data as a predetermined data set. As a supplementary explanation, the physical property information stored in the physical property information database 133 can be updated from an external server device via a wired or wireless communication network as illustrated in FIG. 1A described above.

The preprocessing unit 14 includes a category value conversion unit 141, a missing value processing unit 142, and a data standardization/normalization unit 143. When the training data generated by the training data generation unit 13 does not meet a predetermined requirement as a data set (that is, when it is expected that the estimation model cannot to be appropriately generated in the estimation model generation unit 16), the preprocessing unit 14 performs predetermined missing value processing to the training data (data set) generated by the training data generation unit 13.

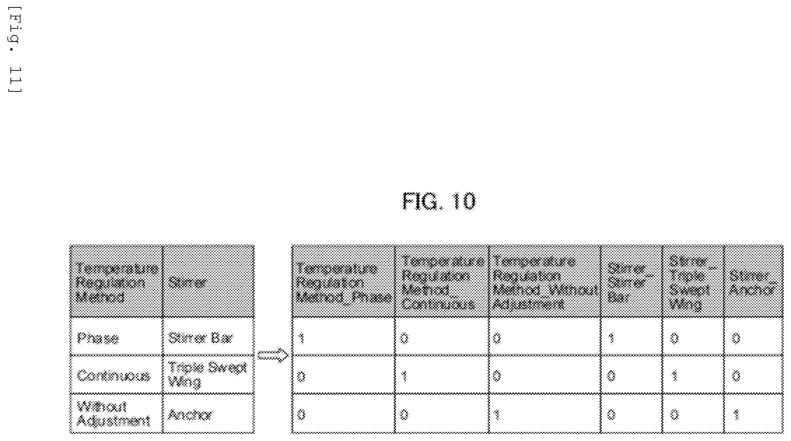

The category value conversion unit 141 converts a category (reactive condition) not indicated as a numerical value into a numerical value. This is because numerical processing by a calculator needs to be executed in the generation of the estimation model, and while details will be described later with reference to FIG. 10, for example, when a “stirrer (triple swept wing)” is set as the reactive condition, the category value conversion unit 141 converts the “stirrer (triple swept wing)” into “0, 1, 0.” As a method for converting the category into the numerical value, Label encoding, One hot encoding, and the like are included.

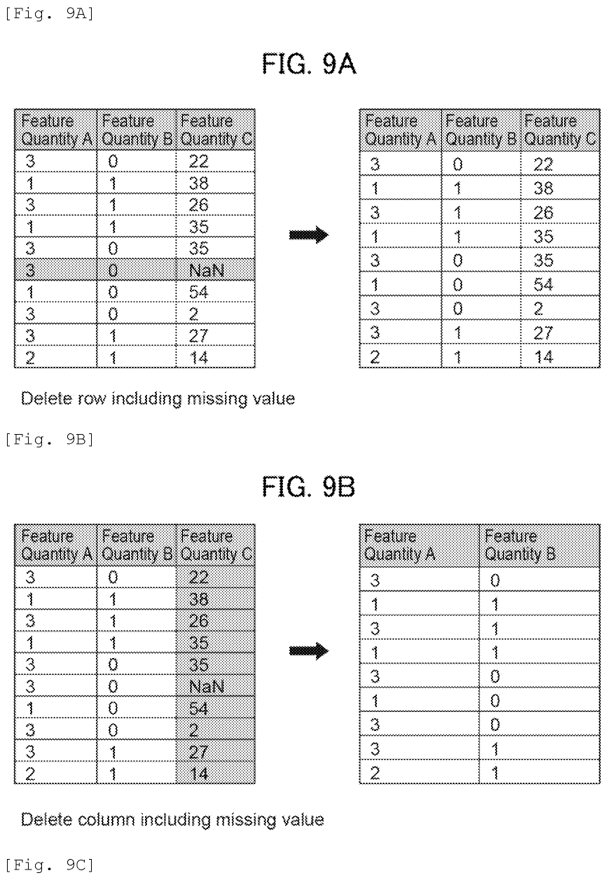

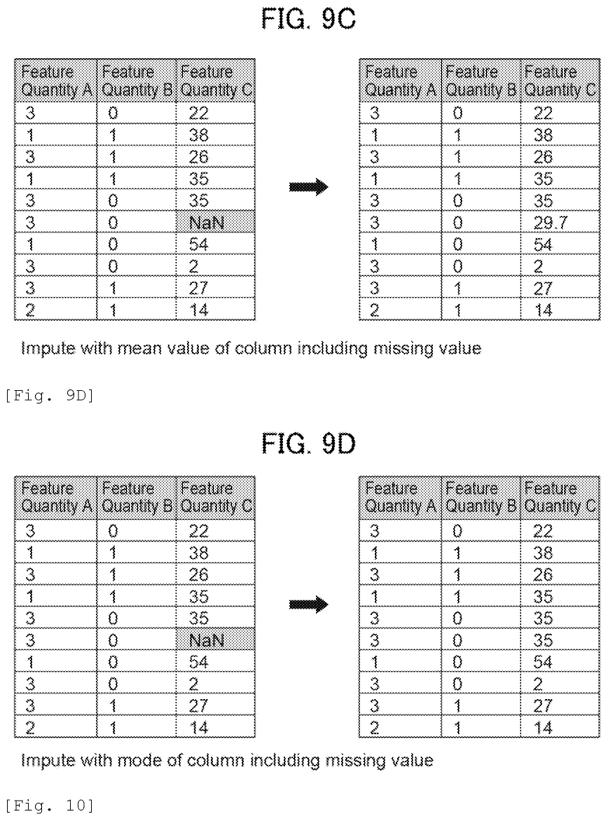

The missing value processing unit 142 executes a process of deleting a part of data or imputing the missing value when the training data (data set) generated by the training data generation unit 13 includes data determined to be missed (a missing value is determined to be present). More specifically, (1) deletion of the row or the column including the missing value in the data set, and (2) imputation of the row or the column including the missing value by a statistic (mean value, median value, mode value, or the like) are executed. The process here will be described later with reference to FIG. 9A to FIG. 9D.

The data standardization/normalization unit 143 standardizes the data, or normalizes the data to a real number value between 0 and 1 using the maximum value/minimum value for each of the feature quantities and the objective variables. Here, as a supplementary explanation, the process by the preprocessing unit 14 is a process performed when the training data generated by the training data generation unit 13 does not meet the predetermined requirement as the data set as described above. When the reactive condition meets the predetermined requirement (that is, for example, when the reactive condition does not need to be converted into the numerical value, when the training data does not include a missing value, or the like), the process by the preprocessing unit 14 does not necessarily need to be performed.

The feature quantity selection unit 15 includes a first feature quantity reduction unit 151, a feature quantity selection model generation unit 152, an importance degree calculation unit 153, a reducibility determination unit 154, a second feature quantity reduction unit 155, and a feature quantity group selection unit 156. The feature quantity selection unit 15 selects the reactive conditions (for example, a reactive condition when the accuracy as the feature quantity selection model is the highest) that meet the predetermined condition as a feature quantity group in generating the estimation model by the estimation model generation unit 16.

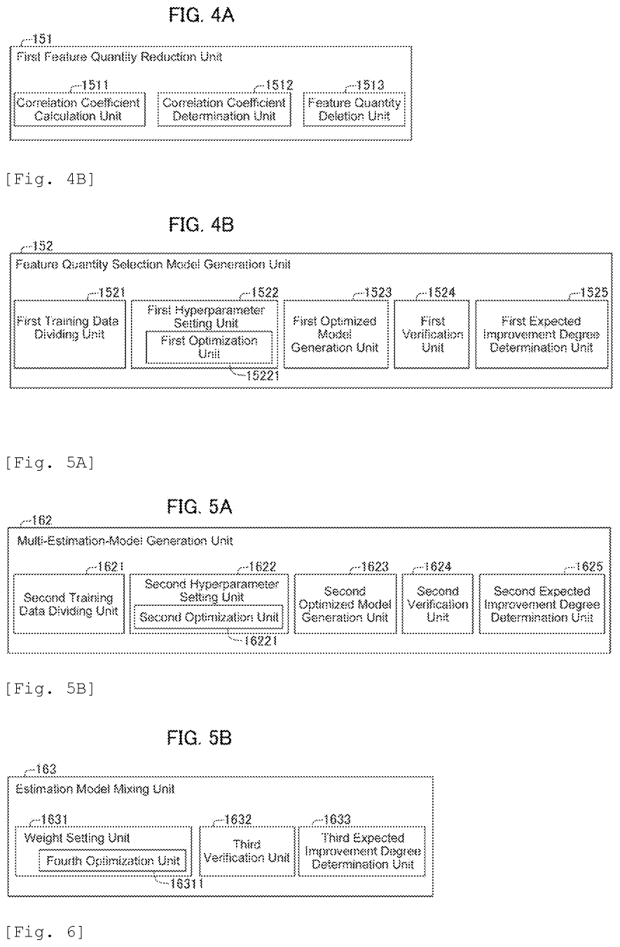

The first feature quantity reduction unit 151 is a function block that executes a process of reducing the feature quantity using a correlation coefficient for avoiding the decrease in prediction accuracy for an unknown input (chemical substance). As illustrated in FIG. 4A, the first feature quantity reduction unit 151 includes a correlation coefficient calculation unit 1511, a correlation coefficient determination unit 1512, and a feature quantity deletion unit 1513.

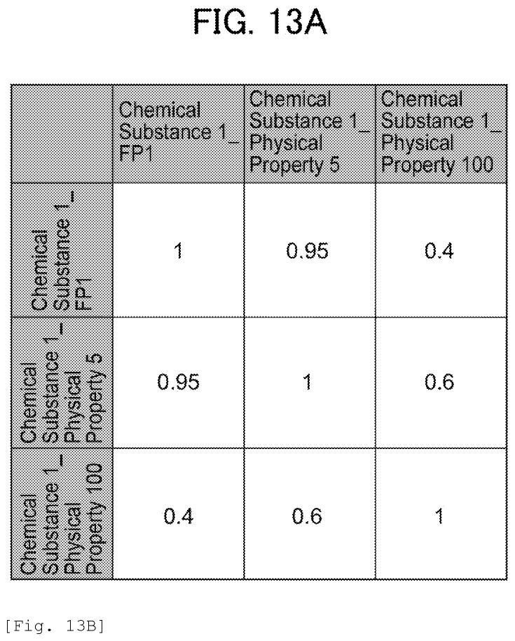

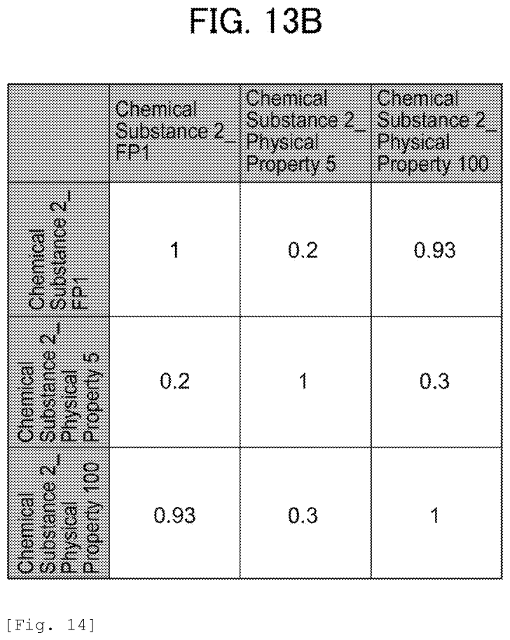

Although described later with reference to FIG. 13A and FIG. 13B, the correlation coefficient calculation unit 1511 calculates a correlation coefficient between the feature quantities (that is, an index for measuring a strength of a linear relation between the two reactive conditions). The correlation coefficient determination unit 1512 determines whether the correlation coefficient calculated by the correlation coefficient calculation unit 1511 is equal to or more than a predetermined threshold or not (more specifically, determines the feature quantity determined to have the correlation value equal to or more than the predetermined threshold as a reduction target). The feature quantity deletion unit 1513 deletes the feature quantity determined as the reduction target by the correlation coefficient determination unit 1512.

The feature quantity selection model generation unit 152 is a function block that executes the process of generating a model for selecting the feature quantity (feature quantity group). As illustrated in FIG. 4B, the feature quantity selection model generation unit 152 includes a first training data dividing unit 1521, a first hyperparameter setting unit 1522, a first optimized model generation unit 1523, a first verification unit 1524, and a first expected improvement degree determination unit 1525.

The first training data dividing unit 1521 divides the training data generated by the training data generation unit 13 (when the preprocessing unit 14 has performed preprocessing (preprocess processing) to the training data generated by the training data generation unit 13, training data to which the preprocessing has been performed) into training data for generating a feature quantity selection model with an optimized hyperparameter and verification data for verifying the generated feature quantity selection model by the first verification unit 1524 at a predetermined proportion. The predetermined proportion here is set, for example, such that the training data for generating the feature quantity selection model is 90, and the verification data for verifying the generated feature quantity selection model is 10. The division here is performed in units of data set, and the same applies to the processes of dividing into the training data and the verification data below.

The first hyperparameter setting unit 1522 sets a hyperparameter for generating a feature quantity selection model in the first optimized model generation unit 1523 in the later stage. The hyperparameter here is a parameter unique to a model, and defined as a parameter that the designer or the model builder needs to set in advance (or a settable parameter).

The first hyperparameter setting unit 1522 includes a first optimization unit for setting the hyperparameter, and for example, causes the first optimization unit to sweep the hyperparameter by using the Bayesian optimization or the like, thus setting the hyperparameter. The first optimization unit outputs an expected improvement degree (outputs Expected Improvement (EI) or the like in the Bayesian optimization) in the setting of the hyperparameter. As a supplementary explanation, in the setting (sweep) of the hyperparameter, a random search, a grid search, or the like can be applied in addition to the Bayesian optimization applied in this embodiment.

When the hyperparameter is set by the first hyperparameter setting unit 1522, the first optimized model generation unit 1523 generates a model (feature quantity selection model) for selecting the feature quantity (feature quantity group) using a machine learning method such as a decision tree, a neural network, a gradient boosting, and a support vector.

The first verification unit 1524 verifies the feature quantity selection model generated by the first optimized model generation unit 1523 using the training data (that is, verification data for verifying the feature quantity selection model) divided by the first training data dividing unit 1521, and generates accuracy information. The generated accuracy information is stored in the storage unit 17 together with the feature quantity (feature quantity group) by the control unit 11.

The first expected improvement degree determination unit 1525 determines whether the hyperparameter needs to be adjusted by the first hyperparameter setting unit 1522 (more precisely, a first optimization unit 15221) or not by comparing the expected improvement degree output by the first optimization unit 15221 with a predetermined threshold. That is, whether there is a room for improving the accuracy of the feature quantity selection model to be generated or not is determined. Then, when it is determined that there is a room for improvement, the control unit 11 performs the control to repeatedly execute the process from the hyperparameter setting process in the first hyperparameter setting unit 1522 to the verification process in the first verification unit 1524 until the first expected improvement degree determination unit 1525 determines that there is no room for improvement.

The importance degree calculation unit 153 calculates an importance degree of the feature quantity for the feature quantity selection model with the accuracy information that meets a predetermined condition (for example, feature quantity selection model with the highest accuracy, one feature quantity selection model among the feature quantity selection models with the accuracy equal to or more than a predetermined threshold, or the like) among the accuracy information on the feature quantity selection models stored by the control unit 11. More specifically, which feature quantity contributes to the accuracy of the feature quantity selection model is quantified. Here, the feature quantity is a feature in machine learning (that is, one quantitatively indicating the feature and/or the property of an object), and here, the chemical structure information and the physical property information derived from the information on a plurality of chemical substances, the chemical structure information and the physical property information derived from the product, and the reactive condition correspond to the feature quantity.

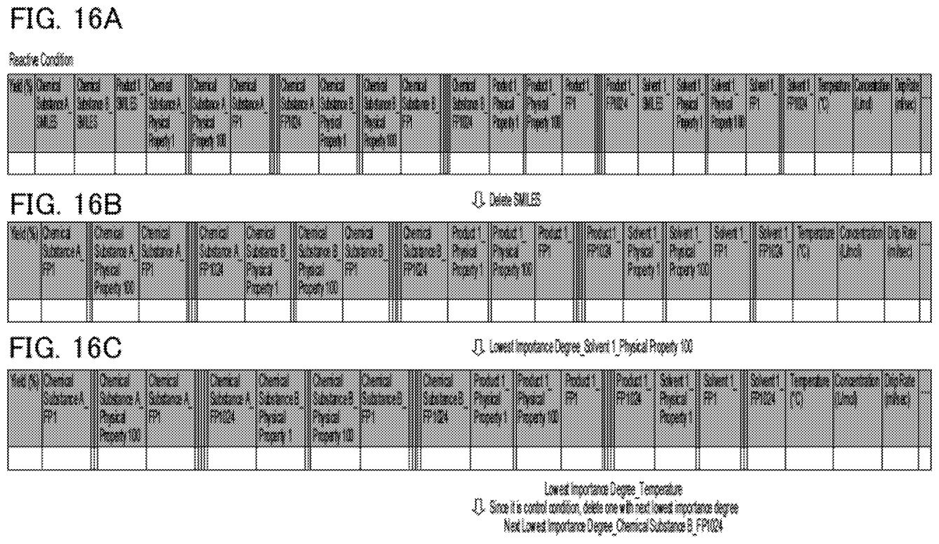

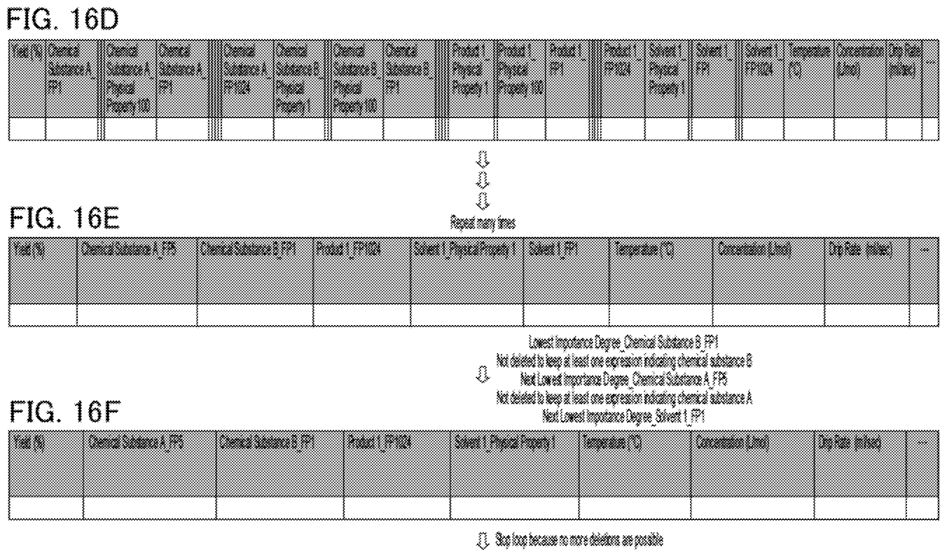

The reducibility determination unit 154 determines whether the feature quantity is deletable or not based on the importance degree calculated by the importance degree calculation unit 153. Basically, the reducibility determination unit 154 determines the feature quantity with the low importance degree among the feature quantities as a deletion target. Exceptionally, when the feature quantity with the low importance degree corresponds to the control condition, the reducibility determination unit 154 determines a feature quantity with the next low importance degree as a deletion target when the feature quantity with the next low importance degree does not correspond to the control condition and a plurality of feature quantities with the next low importance degree are present as the feature quantities without deleting the feature quantity with the low importance degree (that is, corresponding to the property of the feature quantity with the low importance degree, without deleting the feature quantity with the low importance degree). This determination process is repeatedly executed until the feature quantity determined as the deletion target is not included. The process here will be described later with reference to FIG. 16A to FIG. 16F. The second feature quantity reduction unit 155 reduces the feature quantity by deleting the feature quantity determined as the deletion target by the reducibility determination unit 154.

The feature quantity group selection unit 156 selects the feature quantities used for generating the feature quantity selection model with the accuracy information that meets a predetermined condition (for example, feature quantity selection model with the highest accuracy, one feature quantity selection model among the feature quantity selection models with the accuracy equal to or more than a predetermined threshold, or the like) among the stored accuracy information as the feature quantity group every time when the feature quantity selection model is generated. The selected feature quantity group is stored in the storage unit 17 by the control unit 11, and then, transmitted to the estimation model generation unit 16.

The estimation model generation unit 16 includes a third training data dividing unit 161, a multi-estimation-model generation unit 162, and an estimation model mixing unit 163, and generates a plurality of estimation models based on the feature quantity group selected by the feature quantity selection unit 15. The multi-estimation-model generation unit 162 generates a plurality of estimation models according to the respective machine learning methods (decision tree, neural network, gradient boosting, support vector).

The third training data dividing unit 161 randomly divides the training data generated by the training data generation unit 13 (when the preprocessing unit 14 has performed preprocessing (preprocess processing) to the training data generated by the training data generation unit 13, training data to which the preprocessing has been performed) into first training data and first verification data. For example, the random division is performed with the ratio of the first training data to the first verification data of 90 to 10.

As illustrated in FIG. 5A, the multi-estimation-model generation unit 162 includes a second training data dividing unit 1621, a second hyperparameter setting unit 1622, a second optimized model generation unit 1623, a second verification unit 1624, and a second expected improvement degree determination unit 1625, and has a configuration approximately similar to that of the feature quantity selection model generation unit 152. However, the multi-estimation-model generation unit 162 is different in that the multi-estimation-model generation unit 162 generates the estimation model (regression model) according to a plurality of machine learning methods while the feature quantity selection model generation unit 152 generates a model (feature quantity selection model) according to any one of the machine learning methods. The following describes each of the function blocks.

The second training data dividing unit 1621 further divides the first training data divided by the third training data dividing unit 161 into second training data for generating the estimation model with the optimized hyperparameter and second verification data for verifying the generated estimation model by the second verification unit 1624 corresponding to the number of the machine learning used for the modeling. As a method for dividing the first training data into the second training data and the second verification data, for example, a bootstrap method or the like is applicable.

The second hyperparameter setting unit 1622 is implemented corresponding to the number of the machine learning used for the modeling (that is, implemented as a second hyperparameter setting unit 1622-1, a second hyperparameter setting unit 1622-2, . . . a second hyperparameter setting unit 1622-n, and the like), and each of the second hyperparameter setting units 1622 sets the hyperparameter for generating the estimation model in the second optimized model generation unit 1623 corresponding to the machine learning in the later stage.

Similarly to the above description, the hyperparameter here is a parameter unique to a model, and defined as a parameter (settable parameter) that the designer or the model builder needs to set in advance. Each of the second hyperparameter setting units 1622 includes a second optimization unit for setting the hyperparameter, and for example, causes the second optimization unit to sweep the hyperparameter by using the Bayesian optimization or the like, thus setting the hyperparameter. The second optimization unit outputs an expected improvement degree (outputs Expected Improvement (EI) or the like in the Bayesian optimization) in the setting of the hyperparameter.

Similarly to the second hyperparameter setting unit 1622, the second optimized model generation unit 1623 is implemented corresponding to the number of the machine learning used for the modeling (that is, the same number as the number of the second hyperparameter setting units 1622, and implemented as a second optimized model generation unit 1623-1, a second optimized model generation unit 1623-2, . . . a second optimized model generation unit 1623-n, and the like). Each of the second optimized model generation units 1623 is associated with the corresponding second hyperparameter setting unit 1622, and generates the estimation model according to the machine learning method (as described above, for example, decision tree, neural network, gradient boosting, support vector, or the like) set to the second optimized model generation unit 1623 when the hyperparameter is set by the associated second hyperparameter setting unit 1622.

Similarly to the second hyperparameter setting unit 1622, the second verification unit 1624 is implemented corresponding to the number of the machine learning used for the modeling (that is, the same number as the number of the second optimized model generation unit 1623, and implemented as a second verification unit 1624-1, a second verification unit 1624-2, . . . a second verification unit 1624-n, and the like). Each of the second verification units 1624 is associated with the corresponding second optimized model generation unit 1623. When an optimized model (estimation model) is generated by the associated second optimized model generation unit 1623, the second verification unit 1624 verifies the accuracy of the estimation model generated by the associated second optimized model generation unit 1623 using the corresponding second verification data for the validation divided by the second training data dividing unit 1621, and generates the accuracy information. The generated accuracy information is stored in the storage unit 17 together with the estimation model by the control unit 11.

Similarly to the second hyperparameter setting unit 1622, the second expected improvement degree determination unit 1625 is implemented corresponding to the number of the machine learning used for the modeling (that is, the same number as the number of the second verification units 1624, and implemented as a second expected improvement degree determination unit 1625-1, a second expected improvement degree determination unit 1625-2, . . . a second expected improvement degree determination unit 1625-n, and the like). Each of the second expected improvement degree determination units 1625 is associated with the corresponding second verification unit 1624 (second hyperparameter setting unit 1622), and determines whether the hyperparameter needs to be adjusted by the corresponding second hyperparameter setting unit 1622 (more precisely, a second optimization unit 16221) or not by comparing the expected improvement degree output by the corresponding second optimization unit 16221 with a predetermined threshold. That is, whether there is a room for improving the accuracy of the estimation model to be generated or not is determined. Then, when it is determined that there is a room for improvement, the control unit 11 performs the control to repeatedly execute the process from the hyperparameter setting process in the second hyperparameter setting unit 1622 to the verification process in the second verification unit 1624 until the second expected improvement degree determination unit 1625 determines that there is no room for improvement.

As illustrated in FIG. 5B, the estimation model mixing unit 163 includes a weight setting unit 1631, a third verification unit 1632, a third expected improvement degree determination unit 1633. The estimation model mixing unit 163 executes a mixture process to the plurality of estimation models generated by the multi-estimation-model generation unit 162, and sets an optimal weight to each of the plurality of estimation models.

The weight setting unit 1631 includes a fourth optimization unit 16311, and for example, causes the fourth optimization unit 16311 to sweep the weights set to the plurality of estimation models generated in the multi-estimation-model generation unit 162 by using the Bayesian optimization or the like, thus adjusting (setting) the weights. The fourth optimization unit outputs an expected improvement degree (outputs Expected Improvement (EI) or the like in the Bayesian optimization) in the setting of the weight.

The third verification unit 1632 multiplies the weights set to the respective plurality of estimation models generated in the multi-estimation-model generation unit 162 by predicted values of the corresponding to estimation models, and further, compares the result calculated by adding them up with the first verification data. The weights set to the respective plurality of estimation models and the comparison result (verification result) are stored in the storage unit 17 in association with the generated estimation model by the control unit 11.

The third expected improvement degree determination unit 1633 determines whether the weight needs to be adjusted by the weight setting unit 1631 (more precisely, the fourth optimization unit 16311) or not by comparing the expected improvement degree output by the fourth optimization unit 16311 with a predetermined threshold. That is, for each of the plurality of estimation models, the weight is multiplied by the predicted value, and whether there is a room for improvement or not is determined for the accuracy of the result of mixing them. Then, when it is determined that there is a room for improvement, the control unit 11 performs the control to repeatedly execute the weight setting process in the weight setting unit 1631 and the verification process in the third verification unit 1632 until the third expected improvement degree determination unit 1633 determines that there is no room for improvement.

The storage unit 17 stores a predetermined program, intermediate data, execution results (data) of the various kinds of processes as described above. For example, the training data (data set) or the like output in the process reducing the feature quantity corresponds to the intermediate data, and for example, the estimation model and the weight or the like associated with the estimation model correspond to the execution result of the various kinds of processes.

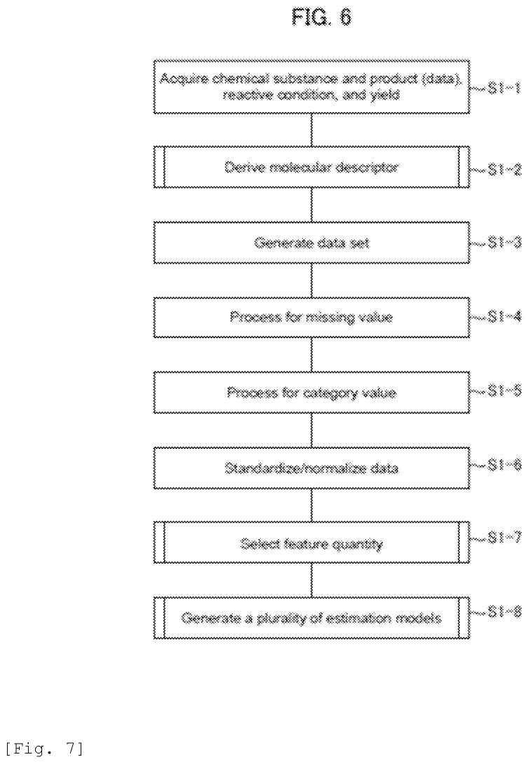

Next, the process of generating the estimation model in the model generation device 1 will be described using the flowchart of FIG. 6. In the following description, a reference sign “S” in the explanation of the flowchart means a step. That is, here, respective processing steps S1-1 to step S1-8 in the flowchart are abbreviated as S1-1 to S1-8. The same applies to flowcharts described below.

In S1-1, the model generation device 1 acquires information on a plurality of chemical substances (information on a chemical substance A described with SMILES notation or the like and information on a chemical substance B described with SMILES notation or the like), information on a product (information on the product described with SMILES notation or the like), a reactive condition, and a yield by the data acquisition unit 12.

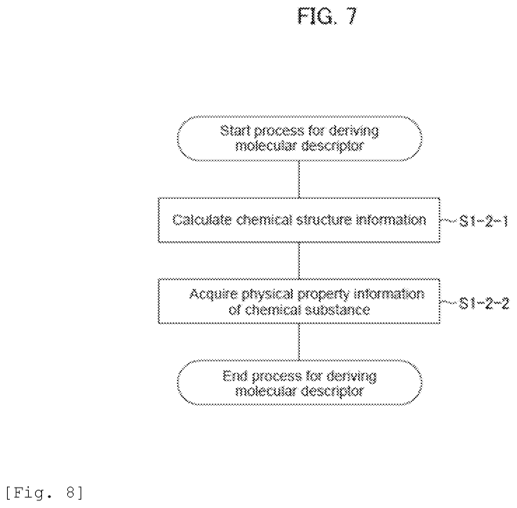

In S1-2, the model generation device 1 derives a molecular descriptor based on the information on the chemical substance by the molecular descriptor derivation unit 132. The process here can be described in detail with reference to FIG. 7. FIG. 7 is a flowchart illustrating a procedure for deriving a molecular descriptor in the molecular descriptor derivation unit 132. In S1-2-1, the molecular descriptor derivation unit 132 calculates the chemical structure information as described above. In S1-2-2, the molecular descriptor derivation unit 132 acquires physical property information from the physical property information database 133 that stores information (physical property information) indicative of physical and/or chemical properties of the chemical substances. The process in S1-2-1 and the process in S1-2-2 may be performed in parallel without considering the order. The model generation device 1 derives a molecular descriptor based on the information on the product similarly to the derivation of the molecular descriptor based on the information on the chemical substance by the molecular descriptor derivation unit 132.

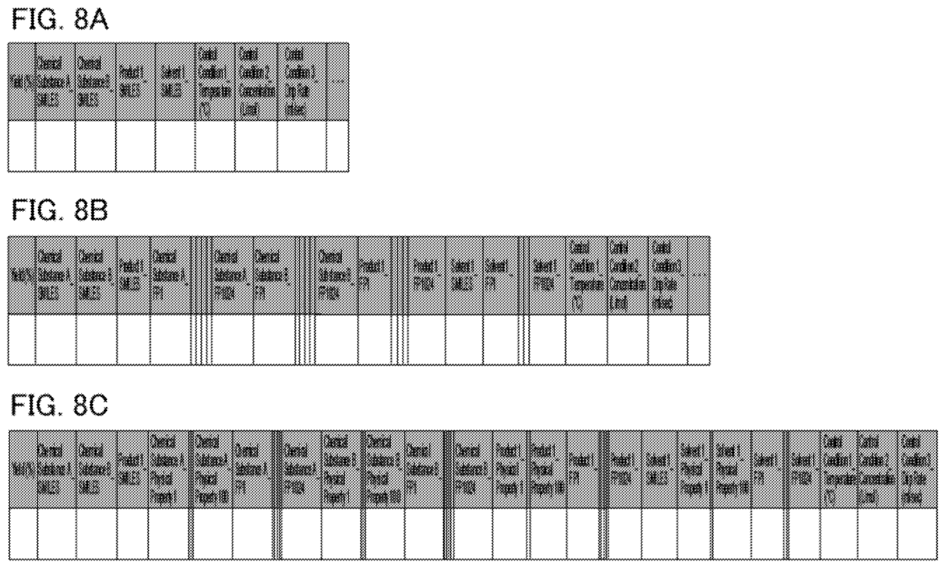

In S1-3, the data set generation unit 131 adds the molecular descriptor derived for the information on the chemical substance and the molecular descriptor derived for the information on the product in S1-2, and the reactive condition and the yield acquired by the data acquisition unit 12 as components constituting the data, thereby generating a data set as illustrated in FIG. 8 as training data.

FIG. 8A, FIG. 8B and FIG. 8C are drawings illustrating a generation process of a data set as an example, and more specifically, illustrating an example of the process of the generation process. FIG. 8A illustrates a first step of the data set generation stage, and illustrates a data set including the yield, the information on the chemical substances (chemical substance A and chemical substance B), the information on the product produced when the chemical substance A is reacted with the chemical substance B, and the information on the reactive conditions (solvent, temperature, concentration, drip rate). The information on the chemical substance and the information on the product are described with SMILES notation, and some of the reactive conditions are indicated as the control conditions.

FIG. 8B illustrates a second step of the data set generation stage, and illustrates a state where the information on the chemical substances (chemical substance A, chemical substance B, and product produced when the chemical substance A is reacted with the chemical substance B, solvent) is expressed as molecular descriptors (converted into molecular descriptors) in the data set generated in the first step, and the molecular descriptors are added to the data set as data. Further, FIG. 8C illustrates a third step of the data set generation stage, and illustrates a state where data acquired for the information on the chemical substances (chemical substance A, chemical substance B, and product produced when the chemical substance A is reacted with the chemical substance B, solvent) from the physical property information DB 133 is added to the data set in the data set generated in the second step.

In S1-4, the model generation device 1 executes missing value processing (that is, a deletion process of a part of data, or an imputation process of the missing value) by the missing value processing unit 142 when a missing value is present in the training data (data set) generated by the training data generation unit 13 in S1-3.

FIG. 9A to FIG. 9D are drawings illustrating missing value processing in the missing value processing unit 142. Particularly, FIG. 9A illustrates a process of deleting a row including a missing value by the row in the data set, FIG. 9B illustrates a process of deleting a column including a missing value by the column in the data set, FIG. 9C illustrates a process of imputation with a statistic (mean value) of a column including a missing value in the data set, and FIG. 9D illustrates a process of imputation with a statistic (mode value) of a column including a missing value in the data set.

In S1-5, when a category (reactive condition) not indicated as a numerical value is present in the training data (data set) generated in S1-3 or the training data (data set) after the missing value processing performed to the training data generated by the training data generation unit 13 in the case where the missing value processing is performed in S1-4, the model generation device 1 converts the category (reactive condition) not indicated as a numerical value into a numerical value by the category value conversion unit 141.

FIG. 10 is a drawing illustrating a process in the category value conversion unit 141, and specifically, illustrates a process of converting a category value into a numerical value using One hot encoding method as an example. In FIG. 10, each of categorical variables of a temperature regulation method and a stirrer is converted into a dummy variable so as to facilitate the learning of an algorithm of machine learning. More specifically, as illustrated in FIG. 10, for the “Temperature Regulation Method,” “Temperature Regulation Method Phase” is converted into “1, 0, 0,” “Temperature Regulation Method Continuous” is converted into “0, 1, 0,” and “Temperature Regulation Method No adjustment” is converted into “0, 0, 1.” For the “Stirrer,” “Stirrer Stirrer Bar” is converted into “1, 0, 0,” “Stirrer Triple Swept Wing” is converted into “0, 1, 0,” and “Stirrer Anchor” is converted into “0, 0, 1.”

In S1-6, the model generation device 1 standardizes data, or normalizes the data to a real number value between 0 and 1 using the maximum value/minimum value of data constituting the category for each of the categories constituting the data set by the data standardization/normalization unit 143.

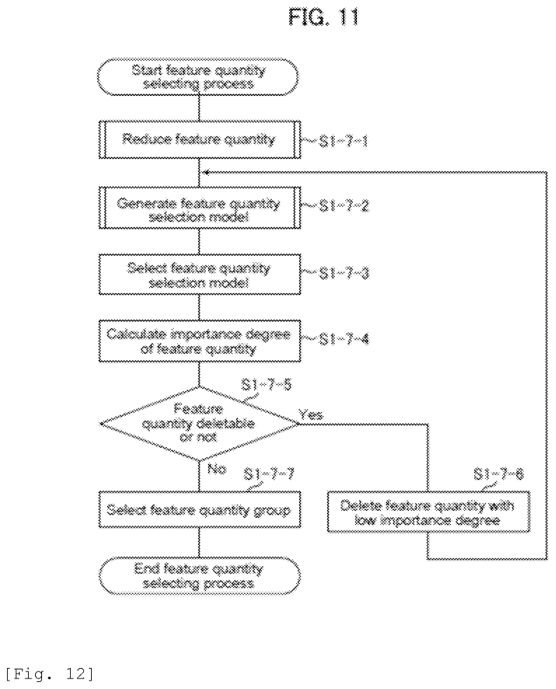

In S1-7, the model generation device 1 selects the reactive conditions of the feature quantity selection model with the accuracy information that meets a predetermined condition (for example, feature quantity selection model with the highest accuracy, one feature quantity selection model among the feature quantity selection models with the accuracy equal to or more than a predetermined threshold, or the like) as a group of the feature quantities (feature quantity group) for generating the estimation model in S1-8 by the feature quantity selection unit 15. The process here can be described in detail with reference to FIG. 11.

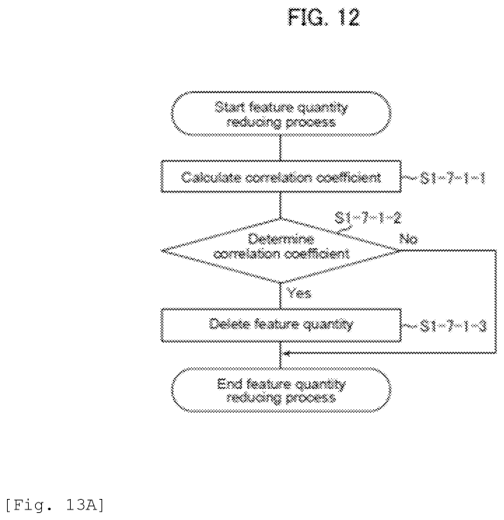

FIG. 11 is a flowchart illustrating a procedure for a process of selecting a feature quantity (feature quantity group) in the feature quantity selection unit 15. In S1-7-1, the model generation device 1 reduces (deletes) the feature quantity in the training data (data set) by the first feature quantity reduction unit 151. The process here can be described in more detail by the flowchart of FIG. 12 as a supplementary explanation.

In S1-7-1-1, the model generation device 1 calculates a correlation coefficient by the correlation coefficient calculation unit 1511 of the first feature quantity reduction unit 151. Here, FIG. 13A and FIG. 13B are drawings illustrating a correlation coefficient and a process of reducing the feature quantity according to the correlation coefficient, and in FIG. 13A and FIG. 13B, correlation coefficients between one feature quantity and the other feature quantity are calculated, and the calculated correlation coefficients are each illustrated at respective corresponding positions in matrixes.

In S1-7-1-2, the model generation device 1 determines one feature quantity of the feature quantities indicated by a predetermined correlation coefficient as a deletion target by the correlation coefficient determination unit 1512 of the first feature quantity reduction unit 151. Here, when the predetermined correlation coefficient is assumed to be 0.9, in FIG. 13A, since the correlation coefficient between a chemical substance 1_FP1 and a chemical substance 1_physical property 5 is calculated to be equal to or more than the predetermined correlation coefficient (correlation value) (that is, calculated to be 0.95, which is 0.9 or more), the correlation coefficient determination unit 1512 determines the chemical substance 1_FP1 and the chemical substance 1_physical property 5 as candidates of the deletion target at first. Note that, here, FP (fingerprint) of the chemical substance 1_FP1 means chemical structure information, and indicates whether a compound has a specific partial structure or not by “0” or “1.”

Next, the correlation coefficient determination unit 1512 determines any of the chemical substance 1_FP1 and the chemical substance 1_physical property 5 as the deletion target based on respective correlation coefficients with another feature quantity (in this case, a chemical substance 1_physical property 100) of the chemical substance 1_FP1 and the chemical substance 1_physical property 5. Specifically, since the correlation coefficient between the chemical substance 1_FP1 and the chemical substance 1_physical property 100 is 0.4, and the correlation coefficient between the chemical substance 1_physical property 5 and the chemical substance 1_physical property 100 is 0.6 (that is, the correlation coefficient (correlation value) between the chemical substance 1_physical property 5 and the chemical substance 1_physical property 100 is higher than the correlation coefficient (correlation value) between the chemical substance 1_FP1 and the chemical substance 1_physical property 100), the correlation coefficient determination unit 1512 determines the chemical substance 1_physical property 5 as the deletion target.

The determination process is performed similarly also in the case of FIG. 13B, and the correlation coefficient determination unit 1512 determines a chemical substance 2_FP1 and a chemical substance 2_physical property 100 as candidates of the deletion target at first, and next, determines the chemical substance 2_physical property 100 as the deletion target based on a correlation coefficient with another feature quantity (in this case, a chemical substance 2_physical property 5).

In S1-7-1-3, the model generation device 1 deletes the feature quantity determined as the deletion target in S1-7-1-2 by the feature quantity deletion unit 1513 of the first feature quantity reduction unit 151. By the process as described above, the feature quantity can be reduced in the training data (data set).

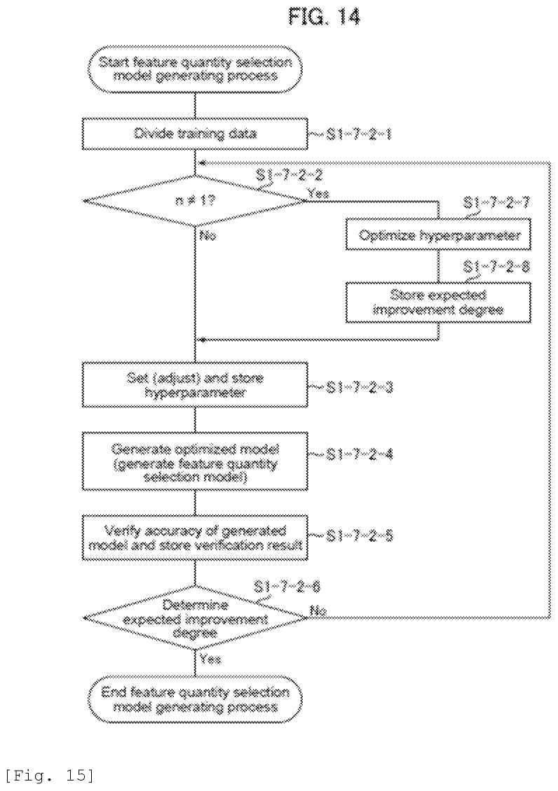

With reference to the flowchart of FIG. 11 again, when the process of reducing the feature quantity in S1-7-1 is performed, the model generation device 1 generates the feature quantity selection model by the feature quantity selection model generation unit 152 of the feature quantity selection unit 15 in S1-7-2. The process here can be described in more detail by the flowchart of FIG. 14 as a supplementary explanation.

In S1-7-2-1, the model generation device 1 divides the training data into training data for generating the feature quantity selection model with an optimized hyperparameter and verification data for verifying the generated feature quantity selection model by the first verification unit 1524 at a predetermined proportion by the first training data dividing unit 1521 of the feature quantity selection model generation unit 152. Each of the divided training data is stored in the storage unit 17 by the control unit 11.

In S1-7-2-2, since the process of generating the feature quantity selection model has not been performed yet, the model generation device 1 determines to be n=1 (S1-7-2-2 No), and transitions the process to S1-7-2-3 by the control unit 11. In S1-7-2-3, the model generation device 1 sets an initial value as the hyperparameter by the first hyperparameter setting unit 1522 of the feature quantity selection model generation unit 152 on the premise that the process of generating the feature quantity selection model has not been performed yet.

In S1-7-2-4, the model generation device 1 generates the feature quantity selection model according to the set hyperparameter (initial value) by the first optimized model generation unit 1523 of the feature quantity selection model generation unit 152. In S1-7-2-5, the model generation device 1 verifies the accuracy of the feature quantity selection model generated in S1-7-2-4 by the first verification unit 1524 of the feature quantity selection model generation unit 152. The model generation device 1 stores the result of the verification (verification result) in the storage unit 17 by the control unit 11.

In S1-7-2-6, the model generation device 1 determines whether to return the process to S1-7-2-2 or not by comparing the expected improvement degree set as the initial value with a predetermined threshold by the first expected improvement degree determination unit 1525 of the feature quantity selection model generation unit 152. Here, the initial value is set such that the models are generated by the number necessary and sufficient for selecting the feature quantity (that is, set such that the process is returned to S1-7-2-2).

When the process is returned to S1-7-2-2, the model generation device 1 determines to be n+1 (S1-7-2-2 Yes) because the process of generating the feature quantity selection model has been performed, and transitions the process to S1-7-2-7 by the control unit 11. In S1-7-2-7, the model generation device 1 executes optimization of the hyperparameter by the first hyperparameter setting unit 1522 (first optimization unit 15221) of the feature quantity selection model generation unit 152. The first hyperparameter setting unit 1522 outputs the expected improvement degree in the process of executing the optimization.

In S1-7-2-8, the model generation device 1 stores the expected improvement degree output in the execution of the optimization process of the hyperparameter in S1-7-2-7 in the storage unit 17 by the control unit 11. Then, the process is transitioned to S1-7-2-3, and the model generation device 1 sets the hyperparameter optimized in S1-7-2-7 as a hyperparameter for generating the optimized model by the first hyperparameter setting unit 1522 of the feature quantity selection model generation unit 152.

In S1-7-2-4, the model generation device 1 generates an optimized model (that is, feature quantity selection model in which the hyperparameter has been optimized) using the hyperparameter optimized in S1-7-2-7 and set in S1-7-2-3 by the first optimized model generation unit 1523 of the feature quantity selection model generation unit 152. In S1-7-2-5, the model generation device 1 verifies the accuracy of the optimized model generated in S1-7-2-4 by the first verification unit 1524 of the feature quantity selection model generation unit 152. The verification result is stored in the storage unit 17 by the control unit 11.

In S1-7-2-6, the model generation device 1 determines whether to return the process to S1-7-2-2 or not by comparing the expected improvement degree output and stored in S1-7-2-8 with a predetermined threshold by the first expected improvement degree determination unit 1525 of the feature 152. More quantity selection model generation unit specifically, the model generation device 1 returns the process to S1-7-2-2 when the expected improvement degree output and stored in S1-7-2-8 is determined to be less than the predetermined threshold, and ends the process of generating the feature quantity selection model when the expected improvement degree output and stored in S1-7-2-8 is determined to be equal to or more than the predetermined threshold by the first expected improvement degree determination unit 1525 of the feature quantity selection model generation unit 152.

Thus, in the process of S1-7-2, the processes of S1-7-2-2, S1-7-2-7, S1-7-2-8, S1-7-2-3, S1-7-2-4, S1-7-2-5, and S1-7-2-6 are repeatedly executed until the expected improvement degree output and stored in S1-7-2-8 becomes equal to or more than the predetermined threshold, and the optimized hyperparameter and the verification result are stored for each execution. Then, when the expected improvement degree output and stored in S1-7-2-8 becomes equal to or more than the predetermined threshold, the process illustrated in FIG. 14 (that is, process of S1-7-2) ends.

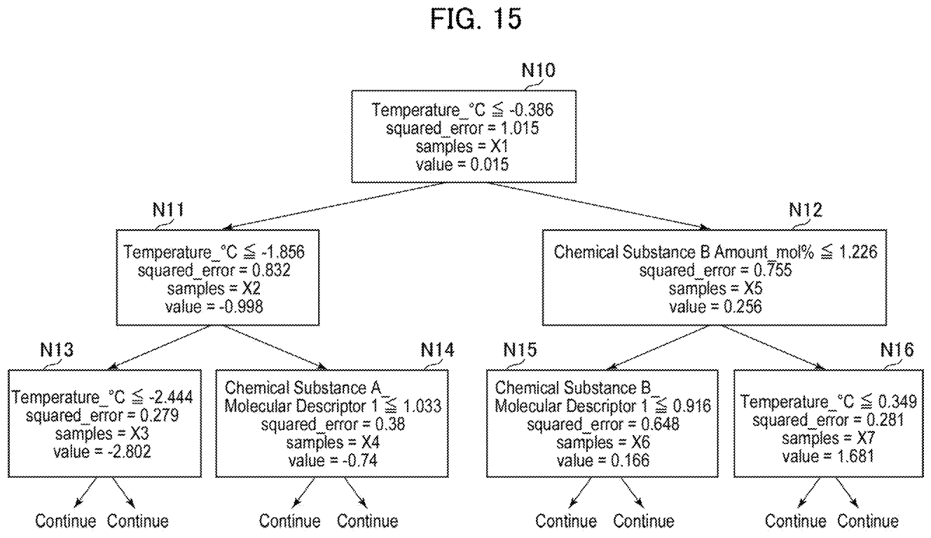

As a supplementary explanation, the feature quantity selection model generated in S1-7-2-4 will be described with an example in which a decision tree is used as the machine learning method. FIG. 15 is a drawing illustrating a model (decision tree model) when a decision tree is used as the machine learning. Here, the decision tree is a model for estimating (classifying) data by branching according to predetermined conditions. FIG. 15 illustrates nodes to which the feature quantities are assigned with a reference numeral N10 to a reference numeral N16 (that is, conditions of the feature quantities (that is, branching conditions) are set to the respective nodes), and illustrates branching in which the nodes are classified to any of lower nodes according to the branching condition.

For example, the node indicated by the reference numeral N10 is branched based on whether to meet temperature_° C.≤−0.386 or not (that is, based on whether the temperature of the reactive condition is −0.386 or less or not). More specifically, in the case of not meeting temperature_° C.≤−0.386 (that is, when the temperature is larger than −0.386), the node of the reference numeral N10 is branched to the node of the reference numeral N11. In the case of meeting temperature_° C.≤−0.386 (that is, when the temperature is −0.386 or less), the node of the reference numeral N10 is branched to the node of the reference numeral N12. The temperature here (that is, −0.386) is indicated as a normalized/standardized value.

In the branching from the node of the reference numeral N10 to the nodes of the reference numeral N11 and the reference numeral N12, the condition of the feature quantity is set such that a sum of mean squared errors becomes minimum, thus performing the branching (division). That is, branching is performed such that the sum of the mean squared error in the node of the reference numeral N11 and the mean squared error of the node of the reference numeral N12 becomes minimum (here, it is determined that the sum (1.587=0.832+0.755) of the mean squared error described in the node of the reference numeral N11 and the mean squared error described in the node of the reference numeral N12 is minimum as the sum of the mean squared errors, and branching is performed with the feature quantity conditions of temperature_° C.≤−1.856 and chemical substance B amount_mol %≤1.226).

In the drawing, Samples described in the node indicates the number of samples after dividing, Value indicates the mean value, and those values are used in the calculation of the mean squared error. Additionally, as a supplementary explanation, in the decision tree illustrated in FIG. 15, the feature quantities of the node of the decision tree are indicated by the temperature, the molecular descriptor, and the like. However, since the branching is performed using the mean squared error, as described above, other reactive conditions (for example, concentration, drip rate, and the like) may be selected as the feature quantity. While the depth of the decision tree is illustrated up to 2 in FIG. 15, the depth of the decision tree is actually provided to be deeper as illustrated as “Continue” in the drawing.

With reference to the flowchart of FIG. 11 again, when the process of generating the feature quantity selection model in S1-7-2 is executed, the control unit 11 identifies (selects) the feature quantity selection model associated with the accuracy information that meets a predetermined condition (for example, information on the highest accuracy, information on one accuracy among the accuracies equal to or more than a predetermined threshold, or the like) among the accuracy information of the feature quantity selection model in which the hyperparameter has been adjusted and saved in S1-7-3.

In S1-7-4, the model generation device 1 calculates the importance degree, in the feature quantity selection model that is stored as one in which the accuracy information meets the predetermined condition by the importance degree calculation unit 153. The importance degree, as described above, indicates which feature quantity contributes to the accuracy of the feature quantity selection model by a numerical value, and calculated by adding the importance degree for each feature quantity in the established feature quantity selection model.

In S1-7-5, the model generation device 1 determines whether the feature quantity can be reduced (deleted) or not by the reducibility determination unit 154 based on the number of the feature quantities, whether the feature quantity corresponds to the control condition, the importance degree of each feature quantity calculated by the importance degree calculation unit 153, and the like. When it is determined that the feature quantity can be reduced (deleted) (S1-7-5 Yes), the model generation device 1 transitions the process to S1-7-6.

In S1-7-6, the model generation device 1 reduces (deletes) the feature quantity determined to be reducible in S1-7-5 by the second feature quantity reduction unit 155. Subsequently, the process is returned to S1-7-2, and the processes of S1-7-6, S1-7-2, S1-7-3, S1-7-4, and S1-7-5 are repeatedly executed until it is determined that the feature quantity cannot be deleted (S1-7-5 No) in S1-7-5.

Here, as a supplementary explanation, with reference to FIG. 16A to FIG. 16F, the reduction process repeatedly performed in the second feature quantity reduction unit 155 will be described. FIG. 16A illustrates the feature quantity selection model associated with the accuracy information that meets a predetermined condition identified (selected) by the control unit 11 (for example, information on the highest accuracy, information on one accuracy among the accuracies equal to or more than a predetermined threshold, or the like) as a data set. In FIG. 16A, when the importance degree calculation unit 153 calculates the importance degree of a solvent 1_physical property 100 to be the lowest importance degree, the reducibility determination unit 154 determines that the feature quantity is reducible because the solvent 1_physical property 100 does not correspond to the control condition, and a solvent 1_physical property 1 is present as the feature quantity (additionally, because a solvent 1_physical property 1 to a solvent 1_physical property 99 are present as the feature quantities).

Next, the solvent 1_physical property 100 calculated to have the lowest importance degree by the importance degree calculation unit 153 and determined as the reduction target by the reducibility determination unit 154 is reduced (deleted) by the second feature quantity reduction unit 155 (FIG. 16B). Subsequently, the process of generating the feature quantity selection model, the process of calculating the importance degree of the feature quantity selection model identified (selected) based on the predetermined condition, and the process of determining whether the feature quantity can be deleted or not are executed again.

In the determination process by the reducibility determination unit 154, whether a deletable feature quantity is included in the feature quantities illustrated in FIG. 16C or not is determined. Here, among the feature quantities illustrated in FIG. 16C, while the feature quantity having the lowest importance degree is the temperature (° C.), the temperature (C) is not determined as the reduction target because the temperature (° C.) corresponds to the control condition. The feature quantity (chemical substance B_FP1024) having the lowest importance degree next to the temperature (° C.) is identified, and further, the chemical substance B_FP1024 is determined as the reduction target under the condition that the feature quantity (chemical substance B_FP1024) does not correspond to the control condition and a plurality of the feature quantities (chemical substance B_FP1024) are present as feature quantities.

When the reducibility determination unit 154 determines that the reduction is performable (determines the chemical substance B_FP1024 as the reduction target), the chemical substance B_FP1024 determined as the reduction target by the reducibility determination unit 154 is reduced (deleted) by the second feature quantity reduction unit 155. Subsequently, the process of generating the feature quantity selection model, the process of calculating the importance degree of the feature quantity selection model identified (selected) based on the predetermined condition, and the process of determining whether the feature quantity can be deleted or not are repeatedly executed until the feature quantity determined as the deletion target is not included (FIG. 16C to FIG. 16F).

With reference to FIG. 11 again, when it is determined that the feature quantity cannot be deleted (S1-7-5 No) in S1-7-5, the model generation device 1 selects a group of the feature quantities remaining without being deleted in S1-7-6 by the feature quantity group selection unit 156 in S1-7-7. When the group of the remaining feature quantities is selected in S1-7-7, a sequence of the processes illustrated in the flowchart of FIG. 11 ends (that is, the process indicated by S1-7 ends).

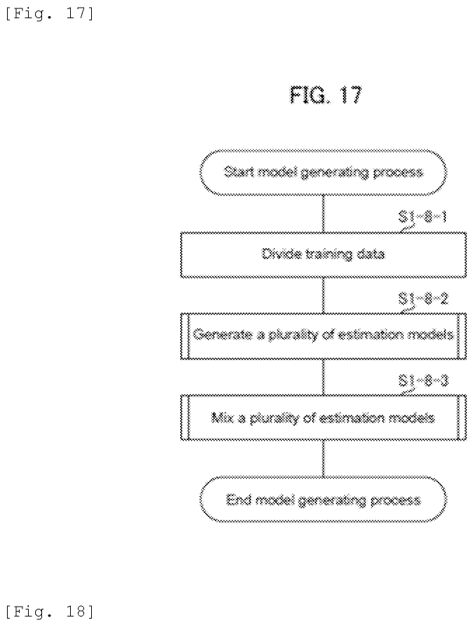

With reference to FIG. 6 again, in S1-8, the model generation device 1 generates a plurality of estimation models by the estimation model generation unit 16. The process here can be described in detail with reference to FIG. 17. FIG. 17 is a flowchart illustrating a procedure for a process of generating a plurality of estimation models by the estimation model generation unit 16.

As illustrated in the flowchart of FIG. 17, in S1-8-1, the model generation device 1 divides the training data generated by the training data generation unit 13 (when the preprocessing unit 14 has performed preprocessing (preprocess processing) to the training data generated by the training data generation unit 13, training data to which the preprocessing has been performed) into first training data used in S1-8-2 and first verification data used in S1-8-3 by the third training data dividing unit 161.

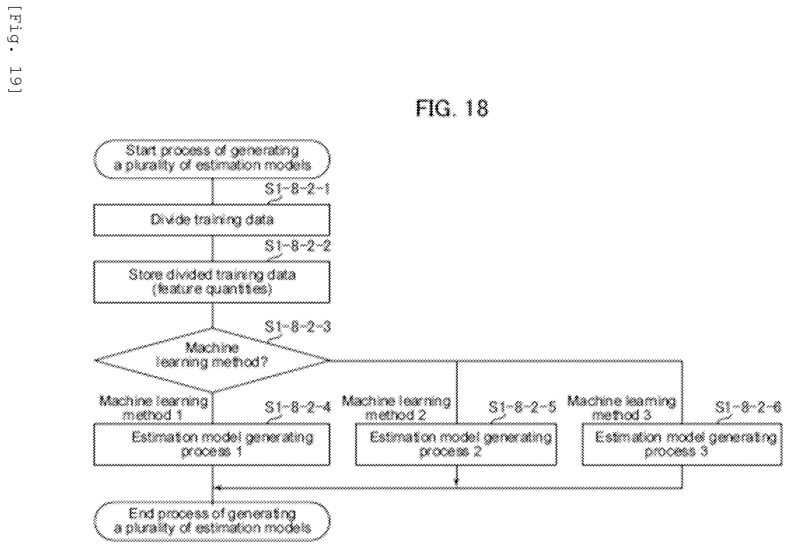

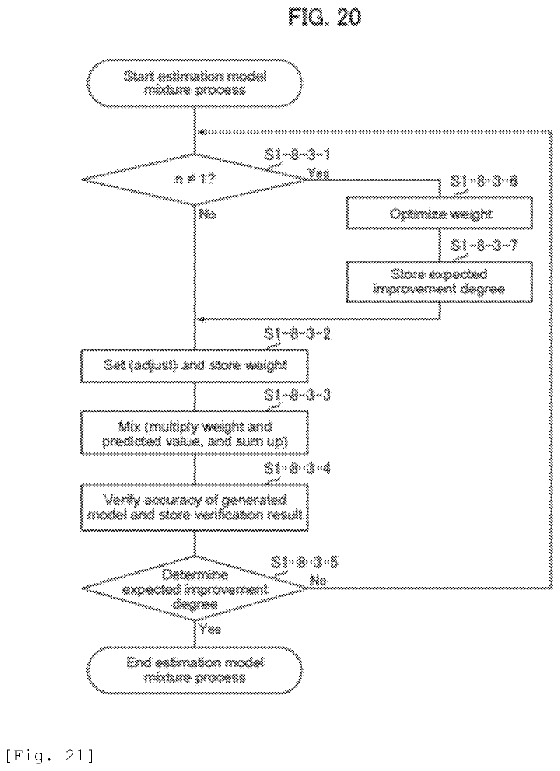

In S1-8-2, a plurality of estimation models are generated by the multi-estimation-model generation unit 162 of the estimation model generation unit 16, and next, in S1-8-3, the plurality of estimation models are mixed to calculate weights set to the respective plurality of estimation models by the estimation model mixing unit 163 of the estimation model generation unit 16. The processes (processes indicated by S1-8-2 and S1-8-3) illustrated in the flowchart of FIG. 17 will be sequentially described with reference to FIG. 18 and FIG. 20, respectively.

FIG. 18 is a flowchart illustrating a procedure for a process (that is, a process for generating a plurality of estimation models) indicated by S1-8-2. In S1-8-2-1, the model generation device 1 divides the first training data divided in S1-8-1 into the second training data for generating the estimation model with the optimized hyperparameter and the second verification data for verifying the generated estimation model in the second verification unit 1624 with a dividing method such as a bootstrap method by the second training data dividing unit 1621 of the multi-estimation-model generation unit 162 (more specifically, here, the first training data is divided into three sets having the second training data for generating the estimation model with the c optimized hyperparameter and the second verification data for verifying the generated estimation model in one set).

In S1-8-2-2, the model generation device 1 stores the second training data for generating the estimation model with the optimized hyperparameter and the second verification data for verifying the generated estimation model, which has been divided into three sets, in the storage unit 17 in the unit of the feature quantity for each set by the control unit 11. In S1-8-2-3, the model generation device 1 assigns the second training data and the second verification data divided as one set to a preliminarily set machine learning method by the control unit 11. As described above, since the first training data is divided into three sets by the bootstrap method, the control unit 11 assigns the three sets to respective machine learning methods 1 to 3 (that is, machine learning method 1, machine learning method 2, and machine learning method 3).

Each of the machine learning methods 1 to 3 is set to any of the machine learning methods such as a decision tree, a neural network, a gradient boosting, and a support vector. In S1-8-2-4, the estimation model is generated with the assigned second training data for generating the estimation model as input data using the set machine learning method. Also in S1-8-2-5 and S1-8-2-6, similarly to S1-8-2-4, the estimation model is generated with the assigned second training data for generating the estimation model as input data using the set machine learning method.

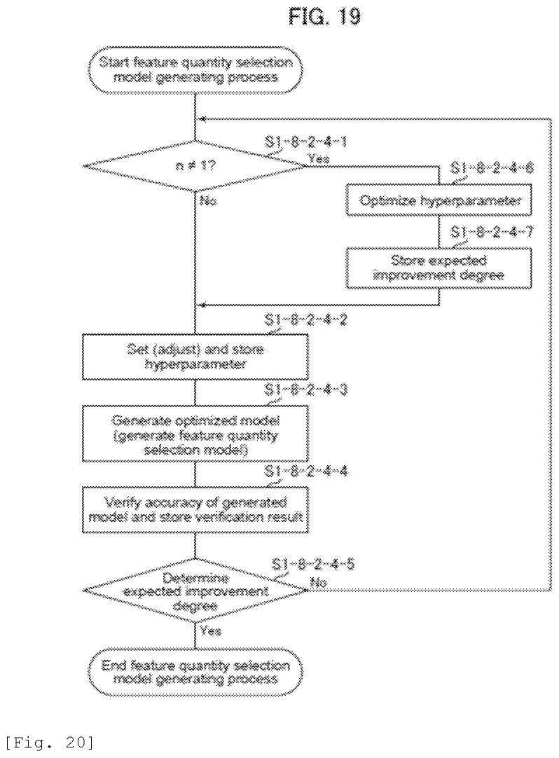

The same machine learning method may be used for the process in S1-8-2-4, the process in S1-8-2-5, and the process in S1-8-2-6. The order of the processes does not matter, and the processes may be performed in parallel. Furthermore, these processes can be described in more detail with reference to FIG. 19, and here, the process in S1-8-2-4 will be described as an example.

In S1-8-2-4-1, since the process of generating the estimation model corresponding to the set machine learning method has not been performed yet, the model generation device 1 determines to be n=1 (S1-8-2-4-1 No), and transitions the process to S1-8-2-4-2 by the control unit 11. In S1-8-2-4-2, the model generation device 1 sets the initial value as the hyperparameter by the second hyperparameter setting unit 1622 of the multi-estimation-model generation unit 162 on the premise that the process of generating the estimation model corresponding to the set machine learning method has not been performed yet.

In S1-8-2-4-3, the model generation device 1 generates the estimation model according to the set hyperparameter (initial value) by the second optimized model generation unit 1623 of the multi-estimation-model generation unit 162. In S1-8-2-4-4, the model generation device 1 verifies the accuracy of the estimation model generated in S1-8-2-4-3 by the second verification unit 1624 of the multi-estimation-model generation unit 162. The verified result (verification result) is stored in the storage unit 17 by the control unit 11.

In S1-8-2-4-5, the model generation device 1 determines whether to return the process to S1-8-2-4-1 or not by comparing the expected improvement degree set as the initial value with a predetermined threshold by the second expected improvement degree determination unit 1625 of the multi-estimation-model generation unit 162. Here, the initial value is set such that the necessary and sufficient number of models are generated for providing the accuracy of the estimation model equal to or more than a predetermined accuracy (that is, set such that the process is returned to S1-8-2-4-1).

When the process is returned to S1-8-2-4-1, since the process of generating the estimation model corresponding to the set machine learning method has been executed, the model generation device 1 determines to be n+1 (S1-8-2-4-1 Yes), and transitions the process to S1-8-2-4-6 by the control unit 11. In S1-8-2-4-6, the model generation device 1 executes the optimization of the hyperparameter by the second hyperparameter setting unit 1622 (second optimization unit 16221) of the multi-estimation-model generation unit 162.

In S1-8-2-4-7, the model generation device 1 stores the expected improvement degree output in the process of executing the optimization process of the hyperparameter in S1-8-2-4-6 in the storage unit 17 by the control unit 11. Subsequently, the process is transitioned to S1-8-2-4-2, and the model generation device 1 sets the hyperparameter optimized in S1-8-2-4-6 to the hyperparameter for generating the estimation model by the second hyperparameter setting unit 1622 of the multi-estimation-model generation unit 162.

In S1-8-2-4-3, the model generation device 1 generates the optimized model (that is, estimation model in which the hyperparameter has been optimized) using the hyperparameter optimized in S1-8-2-4-6 and set in S1-8-2-4-2 by the second optimized model generation unit 1623 of the multi-estimation-model generation unit 162. In S1-8-2-4-4, the model generation device 1 verifies the accuracy of the optimized model generated in S1-8-2-4-3 by the second verification unit 1624 of the multi-estimation-model generation unit 162. The verification result is stored in the storage unit 17 by the control unit 11.

In S1-8-2-4-5, the model generation device 1 determines whether to return the process to S1-8-2-4-1 or not by comparing the expected improvement degree output and stored in S1-8-2-4-7 with a predetermined threshold by the second expected improvement degree determination unit 1625 of the multi-estimation-model generation unit 162. More specifically, the model generation device 1 returns the process to S1-8-2-4-1 when the expected improvement degree output and stored in S1-8-2-4-7 is determined to be less than the predetermined threshold, and ends the process of generating the estimation model when the expected improvement degree output and stored in S1-8-2-4-7 is determined to be equal to or more than the predetermined threshold by the second expected improvement degree determination unit 1625 of the multi-estimation-model generation unit 162.

Thus, in the process of S1-8-2-4, the processes of S1-8-2-4-1, S1-8-2-4-6, S1-8-2-4-7, S1-8-2-4-2, S1-8-2-4-3, S1-8-2-4-4, and S1-8-2-4-5 are repeatedly executed until the expected improvement degree output and stored in S1-8-2-4-7 becomes to be equal to or more than the predetermined threshold. Then, the optimized hyperparameter and the verification result are stored for each execution. With reference to the flowchart of FIG. 18 again, when the process in S1-8-2-5 and the process in S1-8-2-6 are executed similarly to the process in S1-8-2-4, the process of generating a plurality of estimation models illustrated in FIG. 18 ends (that is, the process indicated by S1-8-2 ends).

Next, the process indicated by S1-8-3 (that is, process of mixing the plurality of estimation models) will be described by referring to the flowchart illustrated in FIG. 20. In S1-8-3-1, since the weight setting process has not been performed yet, the model generation device 1 determines to be n=1 (S1-8-3-1 No), and transitions the process to S1-8-3-2 by the control unit 11. In S1-8-3-2, the model generation device 1 sets initial values as the weights added to the respective plurality of estimation models generated by the multi-estimation-model generation unit 162 by the weight setting unit 1631 of the estimation model mixing unit 163 on the premise that the weight setting process has not been performed yet.

In S1-8-3-3, the model generation device 1 multiplies the weights (initial values) set to the respective plurality of estimation models generated by the multi-estimation-model generation unit 162 by predicted values of the corresponding estimation models, and further, adds them up by the third verification unit 1632 of the estimation model mixing unit 163.

In S1-8-3-4, the model generation device 1 compares the execution result (calculation result) of S1-8-3-3 with the first verification data by the third verification unit 1632 of the estimation model mixing unit 163. Then, the model generation device 1 stores the comparison result (verification result) and the weights set to the respective plurality of estimation models in the storage unit 17 in association with the generated estimation models by the control unit 11.

In S1-8-3-5, the model generation device 1 determines whether to return the process to S1-8-3-1 or not by comparing the expected improvement degree set as the initial value with a predetermined threshold by the third expected improvement degree determination unit 1633 of the estimation model mixing unit 163. Here, the initial value is set such that the weight set to each of the plurality of estimation models is added as an optimal value (that is, for increasing the prediction accuracy, set such that the process is returned to S1-8-3-1).

When the process is returned to S1-8-3-1, the model generation device 1 determines to be n+1 (S1-8-3-1 Yes) because the weight setting process has been performed, and transitions the process to S1-8-3-6 by the control unit 11. In S1-8-3-6, the model generation device 1 performs optimization of the weights added to the respective plurality of estimation models by the weight setting unit 1631 (fourth optimization unit 16311) of the estimation model mixing unit 163.

In S1-8-3-7, the model generation device 1 stores the expected improvement degree output in the process of performing the weight optimization process in S1-8-3-6 in the storage unit 17 by the control unit 11. Subsequently, the process is transitioned to S1-8-3-2, and the model generation device 1 sets the weights optimized in S1-8-3-6 as the weights added to the respective plurality of estimation models generated by the multi-estimation-model generation unit 162 by the weight setting unit 1631 of the estimation model mixing unit 163.

In S1-8-3-3, the model generation device 1 multiplies the weights optimized in S1-8-3-6 and set in S1-8-3-2 by the predicted values of the corresponding estimation models, and further, adds them up by the third verification unit 1632 of the estimation model mixing unit 163.

In S1-8-3-4, the model generation device 1 compares the execution result (calculation result) of S1-8-3-3 with the first verification data by the third verification unit 1632 of the estimation model mixing unit 163. Then, the model generation device 1 stores the comparison result (verification result) and the weights set to the respective plurality of estimation models in the storage unit 17 in association with the generated estimation models by the control unit 11.

In S1-8-3-5, the model generation device 1 determines whether to return the process to S1-8-3-1 or not by comparing the expected improvement degree output and stored in S1-8-3-7 with a predetermined threshold by the third expected improvement degree determination unit 1633 of the estimation model mixing unit 163. More specifically, the model generation device 1 returns the process to S1-8-3-1 when the expected improvement degree output and stored in S1-8-3-7 is determined to be less than the predetermined threshold by the third expected improvement degree determination unit 1633 of the estimation model mixing unit 163.

Thus, in the process of S1-8-3-5, the processes of S1-8-3-1, S1-8-3-6, S1-8-3-7, S1-8-3-2, S1-8-3-3, S1-8-3-4, and S1-8-3-5 are repeatedly executed until the expected improvement degree output and stored in S1-8-3-7 becomes to be equal to or more than the predetermined threshold. Then, the optimized weight and verification result are stored in the storage unit 17 in association with the generated estimation model for each execution. When the expected improvement degree output and stored in S1-8-3-7 is determined to be equal to or more than the predetermined threshold in the process of S1-8-3-5, the process of mixing the plurality of estimation models ends (that is, the process indicated by S1-8-3 ends).

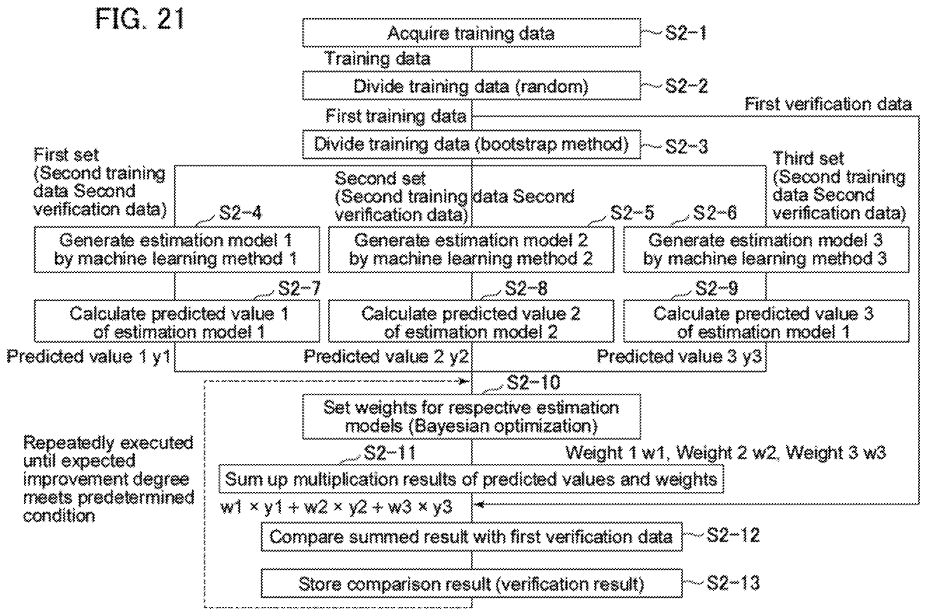

As a supplementary explanation, with reference to FIG. 21, the contents of the process in the estimation model generation unit 16 will be described while focusing on the flow of the data (data flow). In S2-1, the training data generated by the training data generation unit 13 (when the preprocessing unit 14 has performed preprocessing (preprocess processing) to the training data generated by the training data generation unit 13, training data to which the preprocessing has been performed) is acquired by the estimation model generation unit 16.

In S2-2, the acquired training data is divided into the first training data and the first verification data. In S2-3, the divided first training data is further divided into three sets, in which one set includes the second training data and the second verification data, by the bootstrap method. In the division here, duplication of data is allowed. That is, for the second training data divided as the first set and the second training data divided as the second set, the data may be duplicated.

In S2-4, an estimation model 1 is generated by the machine learning method 1 based on the first set (the second training data and the second verification data as the first set). Then, when the estimation model 1 is generated, in S2-7, a predicted value y1 of the estimation model 1 is calculated. Similarly, an estimation model 2 is generated by the machine learning method 2 in S2-5, and a predicted value y2 of the estimation model 2 is calculated in S2-8. An estimation model 3 is generated by the machine learning method 3 in S2-6. A predicted value y3 of the estimation model 3 is calculated in S2-9.

In S2-10, for each of the plurality of estimation models, the weight to the estimation model is set (specifically, a weight w1 to the estimation model 1, a weight w2 to the estimation model 2, and a weight w3 to the estimation model 3 are set). In S2-11, the predicted values of the estimation models are multiplied by the weights corresponding to the estimation models, and the sum total of them is calculated (specifically, a sum total of a product of a predicted value 1 (y1) of the estimation model 1 and a weight 1 (w1) corresponding to the estimation model 1, a product of a predicted value 2 (y2) of the estimation model 2 and a weight 2 (w2) corresponding to the estimation model 2, and a product of a predicted value 3 (y3) of the estimation model 3 and a weight 3 (w3) corresponding to the estimation model 3 is calculated).

In S2-12, the sum total (result) calculated in S2-11 is compared with the first verification data divided in S2-2, thereby verifying the accuracy. In S2-13, the comparison result (verification result) is stored in the storage unit 17 together with the weights set to the respective plurality of estimation models in association with the generated estimation models. The data processing here from S2-10 to S2-13 is repeatedly executed until the expected improvement degree output in the executing process of the Bayesian optimization becomes to be equal to or more than the predetermined threshold as described in the flowchart illustrating the procedure for the above-described process in the estimation model generation unit 16. This is indicated by a dashed line in FIG. 21.

When the estimation model is provided, the estimation model 1 generated in S2-4, the estimation model 2 generated in S2-5, and the estimation model 3 generated in S2-6, and the weight 1, the weight 2, and the weight 3 corresponding to the estimation models, respectively, which are stored in S2-13 and verified as those having the highest accuracy among the verification results corresponding to the estimation models, are provided.

Accordingly, the process indicated by S1-8 in the flowchart of FIG. 6 completes. While the procedure for the process of generating the estimation model in the model generation device 1 has been described above with reference to the flowchart of FIG. 6, for the procedure (process) for the processing of generating the estimation model in the model generation device 1, the processes can be further collectively described. Specifically, the process of deriving the molecular descriptor in S1-2 and the process of generating the data set in S1-3 can be performed together as the process of generating the training data, and the process for the missing value in S1-4, the process for the category value in S1-5, and the process for the standardization/normalization of data in S1-6 can be performed together as the preprocessing.

Additionally, the processes described as the preprocessing (that is, processes indicated by S1-4, S1-5, and S1-6) are the processes executed corresponding to the generated training data (for example, when the training data includes a missing value, the missing value processing (as described above, for example, the process of deleting a part of data, or a process of imputing the missing value) is executed), and are the processes not necessarily executed.

In the flowchart (this embodiment) of FIG. 6, in the model generation, for improving the accuracy, (1) the process for deleting the feature quantity (S1-7-4, S1-7-6, and the like), (2) the process for adjusting the hyperparameter (S1-7-2-7 and the like), and (3) the process for generating a plurality of estimation models and setting the weights (S1-8) are performed. Specifically, the process of adjusting the hyperparameter and the process of deleting the feature quantity to the estimation model generated with the adjusted hyperparameter are repeatedly executed until the predetermined condition is met, and then, the generation of the plurality of estimation models and the setting of the weights are performed using the feature quantity (feature quantity group) used for generating the estimation model with the high accuracy obtained as the result of executing the process for deleting the feature quantity.

Therefore, in S1-7-2-4, the feature quantity selection model generated with the hyperparameter (initial value) can be provided as the estimation model generated by the model generation device 1 on the premise that the appropriate accuracy can be ensured for the feature quantity selection model generated according to the hyperparameter (initial value).

Additionally, it is also possible to individually and independently execute (1) the process for deleting the feature quantity and (2) the process for adjusting the hyperparameter, and provide the feature quantity selection model with the accuracy information that meets a predetermined condition (for example, feature quantity selection model with the highest accuracy, one feature quantity selection model among the feature quantity selection models with the accuracy equal to or more than a predetermined threshold, or the like) among the feature quantity selection models generated by the execution as the estimation model generated by the model generation device 1.

Furthermore, similarly, it is also possible to execute (3) the process for generating a plurality of estimation models and setting the weights individually and independently from (1) the process for deleting the feature quantity and (2) the process for adjusting the hyperparameter. That is, the generation of the plurality of estimation models and the setting of the weights to the respective plurality of generated estimation models can be performed according to the feature quantity of the feature quantity selection model generated with the hyperparameter (initial value).

Additionally, as a supplementary explanation, while the information on two chemical substances, the information on the chemical substance A and the information on the chemical substance B, is used as the information on a plurality of chemical substances for the description in the above-described embodiment, the model can be generated using information on three or more chemical substances. When the information on three or more chemical substances is used, for example, the chemical structure information and the physical property information of the chemical substances, such as a chemical substance C, a chemical substance D, . . . and the like in addition to those of the chemical substance A and the chemical substance B, are added as the components constituting the training data generated as the predetermined data set, and it is only necessary to execute the process of selecting the feature quantity (process of deleting the feature quantity) and the process of generating a plurality of estimation models based on the thus configured data set (training data).

While the machine learning is described using the decision tree as an example in the embodiment, a neural network can be used as described above as the machine learning. The neural network includes an input layer, an output layer, and a plurality of intermediate layers positioned between the input layer and the output layer. Each of the intermediate layers includes a plurality of nodes (neurons), and a bias is set for each of the plurality of nodes. Each of the plurality of nodes is connected to the nodes of the intermediate layers as a former stage and a latter stage by edges with predetermined weights.

On the premise of such a configuration, each of the plurality of nodes of each of the intermediate layers constitutes the former intermediate layer, and performs a product-sum operation of input signals from the nodes connected to the node by the edges, the weights set to the edges, and the biases. Furthermore, application of an activation function constitutes the latter intermediate layer and generates output signals to be output to the nodes thereof connected by the edges. The generated output signals are transmitted to the corresponding nodes of the output layer via the edges. As a supplementary explanation, the weights set to the edges are used for multiplication in the product-sum operation, and the biases set to the nodes are used for addition in the product-sum operation.

In the case where this neural network is used, specifically, when training data generated from information on a plurality of chemical substances, reactive conditions when the plurality of chemical substances are reacted, information on a product produced by reacting the plurality of chemical substances, and yields of the product when the reactions are performed under the reactive conditions, which have been acquired as input data in the model generation device 1, is input to the input layer of the neural network, in each of the intermediate layers, as described above, the product-sum operations and the applications of the activation function are performed, and the output signals are generated. The generated output signals are transmitted to the corresponding nodes of the output layer via the edges.

The model generation device 1 calculates a loss between the output signal (yield) acquired from the output layer and a target signal (training data), changes the biases of the nodes and the weights of the edges in the neural network based on the calculation result, calculates a gradient value of the loss, further calculates a product of the gradient value of the loss and a learning rate, and subtracts the value obtained from the biases of the nodes and the weights of the edges in the neural network. Then, the model generation device 1 repeatedly performs the arithmetic processing, thereby optimizing the biases of the nodes and the weights of the edges in the neural network (that is, the model generation device 1 generates an optimized neural network (estimation model)).

As described above, according to this embodiment, by generating the estimation model having the information on the plurality of chemical substances, which are reaction objects, and the information on the product, which is to be produced, as the input values and the reactive condition in which the yield of the product to be produced meets the predetermined condition as the output value, the man-hour for operation can be reduced in the setting of the reactive condition in which the yield meets the predetermined condition.

### (Providing Reactive Condition (Reactive Condition Providing Device))

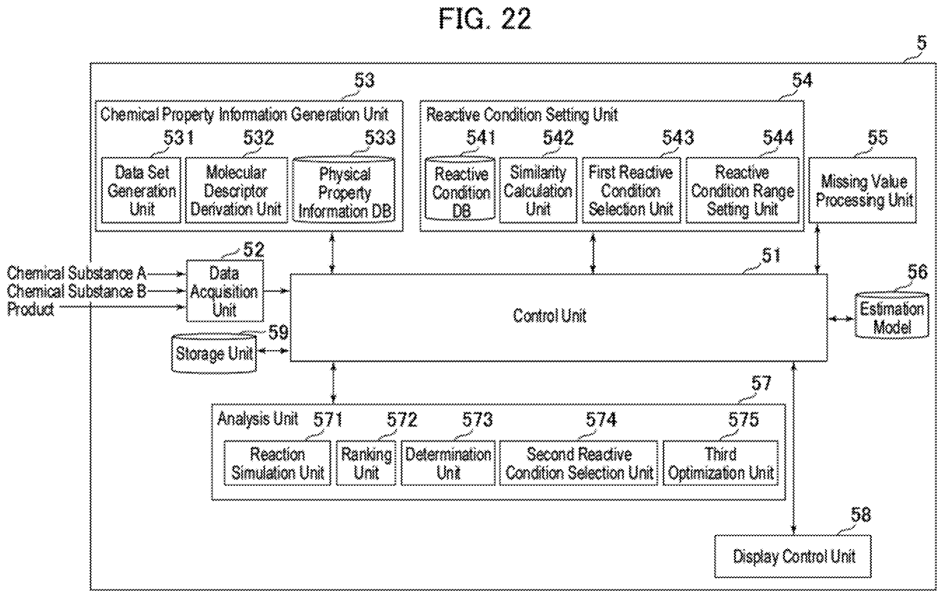

FIG. 22 is a block diagram illustrating a functional configuration of a reactive condition providing device 5 according to the embodiment. As illustrated in FIG. 22, the reactive condition providing device 5 includes, as the functions, mainly, a control unit 51, a data acquisition unit 52, a chemical property information generation unit 53, a reactive condition setting unit 54, a missing value processing unit 55, an estimation model 56, an analysis unit 57, a display control unit 58, and a storage unit 59.

The control unit 51 is a function block that controls processes of the respective function blocks. The data acquisition unit 52 acquires information on a plurality of chemical substances and information on a product (product to be produced) generated by reacting the plurality of chemical substances. The information on the plurality of chemical substances and the information on the product to be produced acquired by the data acquisition unit 52 are temporarily stored in the storage unit 59 by the control unit 51. Note that the information on the chemical substance and the information on the product can be described with the notation such as SMILES notation, MOL file, and SDF file, and in this embodiment, information described with SMILES notation is acquired similarly to the case of the model generation device. Additionally, here, as the information on a plurality of chemical substances, information on two chemical substances, information on a chemical substance A and information on a chemical substance B, is used in the explanation.

The chemical property information generation unit 53 includes a data set generation unit 531, a molecular descriptor derivation unit 532, and a physical property information database (in the drawing, physical property information DB) 533, and generates chemical property information from the information on the chemical substance acquired by the data acquisition unit 52. The data set generation unit 531 generates the chemical property information as a predetermined data set based on chemical structure information derived by the molecular descriptor derivation unit 532 and physical property information acquired from the physical property information database 533. The process executed to the information on the chemical substance here is executed similarly to the information on the product acquired by the data acquisition unit 52. The physical property information stored in the physical property information database 533 can be updated from an external server device via a wired or wireless communication network as illustrated in FIG. 1B described above. The same applies to a reactive condition database 541 described later.

The molecular descriptor derivation unit 532 derives the molecular descriptors from the chemical structure information and the physical property information indicated as indices (numerical values) in determining the molecular structure. In the derivation of the molecular descriptor, similarly to the case of the model generation device, while Morgan method is used in the explanation, Mordred method, RDkit descriptor method, or the like can be used. That is, here, similarly to the case of the model generation device, the chemical structure information is calculated, and the physical property information is acquired from a predetermined database, thereby deriving the molecular descriptor.

The molecular descriptors derived by the molecular descriptor derivation unit 532 (that is, the calculated chemical structure information and the acquired physical property information) are added to the data set as data (components) by the data set generation unit 531. Thus, the data set generation unit 531 adds the molecular descriptor to the information on the chemical substance as a component of the data set, and furthermore, adds the molecular descriptor to the information on the product as a component of the data set, thereby generating the chemical property information as a predetermined data set.

The reactive condition setting unit 54 includes a reactive condition database (in the drawing, reactive condition DB) 541, a similarity calculation unit 542, a first reactive condition selection unit 543, and a reactive condition range setting unit 544. The reactive condition setting unit 54 sets the reactive condition to be input to the analysis unit 57 (more specifically, a reaction simulation unit 571 of the analysis unit 57) based on the chemical property information generated by the chemical property information generation unit 53.

The reactive condition database 541 is a database that stores data on experiments performed in the past. Specifically, a plurality of reacted chemical substances, a reactive condition when the plurality of chemical substances are reacted, and a product produced as a result of the reaction are included in one data set, and the data sets are stored by the number necessary and sufficient for estimating the reactive condition. The plurality of chemical substances and the product stored in the reactive condition database 541 are described with SMILES notation.

The similarity calculation unit 542 calculates a similarity between the information on the plurality of chemical substances acquired by the data acquisition unit 52 and the information on a plurality of chemical substances reacted in the past stored in the reactive condition database 541 using a predetermined index. For the product, similarly, the similarity calculation unit 542 calculates a similarity between the information on the product acquired by the data acquisition unit 52 and the information on the product produced as a result of the reaction in the past using a predetermined index.

Accordingly, by calculating the similarity and inputting the reactive condition selected by evaluating the calculated similarity to the estimation model, the reactive condition with the high yield can be provided (estimated). In identifying a chemical substance similar to (or chemical substance the same as) the input information on the plurality of chemical substances, while the similarity is used as described above in this embodiment, a distance between the chemical substances (distance matrix, similarity matrix) also can be used.

As the index (method) for calculating the similarity, a contingency coefficient, Tanimoto coefficient, Dice coefficient and the like are included, and here, the calculation of the similarity using Tanimoto coefficient will be briefly described as an example. In Tanimoto coefficient, the molecular structure described with SMILES notation is converted into a fingerprint for each of compounds as calculation objects of the similarity, and the similarity between the fingerprints is calculated.

More specifically, a Tanimoto coefficient (here, Tanimoto coefficient between a chemical substance x and a chemical substance y) is indicated as a value obtained by dividing the number of partial structures common in the chemical substance x and the chemical substance y by the total number of the partial structures included in the chemical substance x and the chemical substance y (that is, indicated as a formula below). In the formula, n(x∩y) indicates the number of common partial structures, n(x∪y) indicates the total number (natural number) of the partial structures, and the closer to “1” the divided value is, the higher the similarity between the chemical substance x and the chemical substance y becomes.

$${c}_{Tanimoto} ( x , y ) = \frac{n ( x ⋂ y )}{n ( x ⋃ y )} \qquad [ Math . 1 ]$$

Here, when the chemical substance x and the chemical substance y each have the fingerprints (FP) as illustrated in Table 1 below, n(x∩y) is “4” (FP2, FP3, FP5, FP10), and n(x∪y) is “7” (FP1, FP2, FP3, FP5, FP6, FP8, FP10), and therefore, the Tanimoto coefficient is 4/7 (=0.57). As a reference, Table 2 below illustrates the fingerprints of the chemical substance x and a chemical substance z, and in this case, the Tanimoto coefficient is 5/7 (=0.71). Therefore, when the chemical substance x is an unknown chemical substance, the chemical substance z is determined as a chemical substance more similar to the chemical substance x than the chemical substance y.

TABLE 1

| | FP1 | FP2 | FP3 | FP4 | FP5 | FP6 | FP7 | FP8 | FP9 | FP10 |
|---|---|---|---|---|---|---|---|---|---|---|
| Chemical | 1 | 1 | 1 | 0 | 1 | 0 | 0 | 1 | 0 | 1 |
| Substance x | | | | | | | | | | |
| Chemical | 0 | 1 | 1 | 0 | 1 | 1 | 0 | 0 | 0 | 1 |
| Substance y | | | | | | | | | | |

TABLE 2

| | FP1 | FP2 | FP3 | FP4 | FP5 | FP6 | FP7 | FP8 | FP9 | FP10 |
|---|---|---|---|---|---|---|---|---|---|---|
| Chemical | 1 | 1 | 1 | 0 | 1 | 0 | 0 | 1 | 0 | 1 |
| Substance x | | | | | | | | | | |
| Chemical | 0 | 1 | 1 | 0 | 1 | 1 | 0 | 1 | 0 | 1 |
| Substance y | | | | | | | | | | |

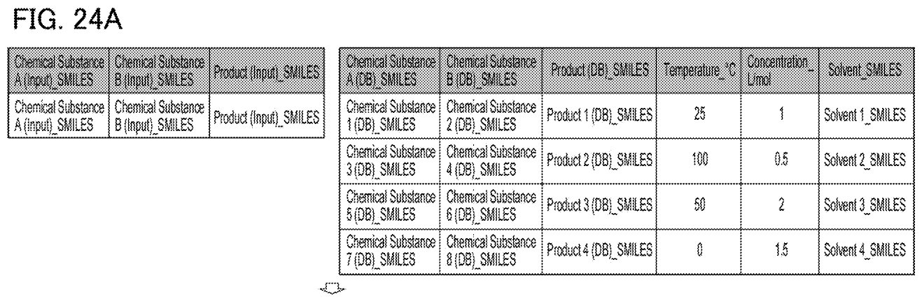

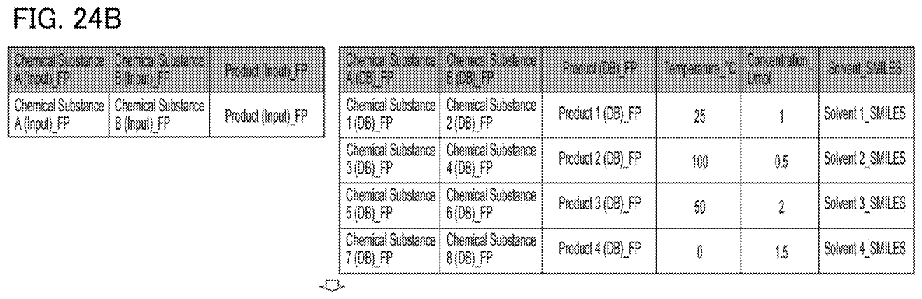

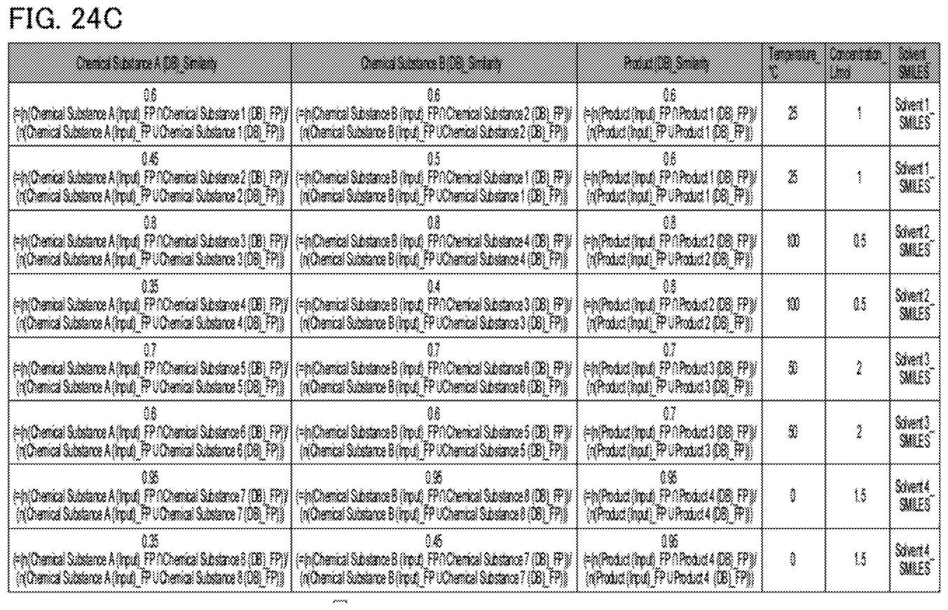

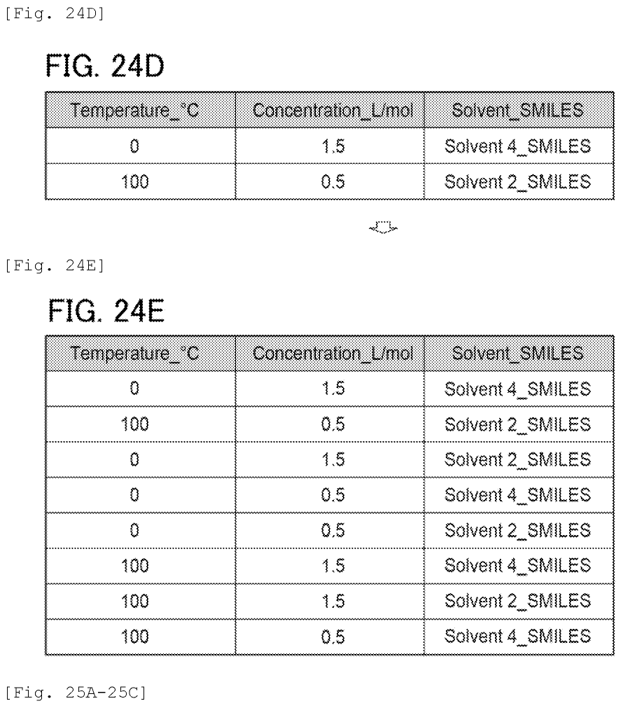

The first reactive condition selection unit 543 selects the reactive conditions by comparing the similarity calculated by the similarity calculation unit 542 with a predetermined threshold. The reactive condition range setting unit 544 sets assumed combinations of the reactive conditions from the selected reactive conditions. The contents of the processes in the similarity calculation unit 542, the first reactive condition selection unit 543, and the reactive condition range setting unit 544 will be specifically described later with reference to FIG. 24A to FIG. 24E.

When the reactive condition set by the reactive condition range setting unit 544 does not meet a predetermined requirement as the data set (that is, when it is expected that the appropriate analysis cannot be performed by the analysis unit 57, and the reactive condition cannot be appropriately output), the missing value processing unit 55 performs predetermined missing value processing to the reactive condition (data set).

In the missing value processing here, similarly to the case of the model generation device, a process of deleting a part of data or imputing (repairing) the missing value is executed. More specifically, (1) deletion of the row or the column including the missing value in the data set, and (2) imputation of the row or the column including the missing value by a statistic (mean value, median value, mode value, or the like) are executed.

The estimation model 56 is a learned estimation model in which machine learning has been preliminarily executed with a machine learning method such as a decision tree, a neural network, a gradient boosting, and a support vector. Here, in this embodiment, it is assumed that the process regarding the learning and the relearning in the estimation model 56 is executed by a device (model generation device 1) different from the reactive condition providing device 5. In this case, for example, the estimation model 56 may be provided to the reactive condition providing device 5 via a predetermined recording medium, or for example, may be delivered to the reactive condition providing device 5 via a wired or wireless communication network by a predetermined server device. The estimation model 56 may be provided to the reactive condition providing device 5 in any aspect. Thus, while an example in which the reactive condition providing device and the model generation device are configured as different devices has been described here, the present invention is not necessarily limited thereto, and a configuration in which one device has a function of a reactive condition providing device and a function of a model generation device may be employed.

The analysis unit 57 includes the reaction simulation unit 571, a ranking unit 572, a determination unit 573, a second reactive condition selection unit 574, and a third optimization unit 575. The analysis unit 57 executes simulations according to the reactive conditions set by the reactive condition setting unit 54, selects the reactive condition under which the yield meets a predetermined condition based on the execution result (analysis result), and sets the selected reactive condition as an output (display) object. When there is no reactive condition under which the yield meets the predetermined condition, the reactive condition optimized by the third optimization unit 575 is set as the output object.

The reaction simulation unit 571 inputs the reactive condition set by the reactive condition range setting unit 544 (when the missing value processing has been executed, the reactive condition in which the missing value processing has been performed to the reactive condition set by the reactive condition range setting unit 544) to the estimation model 56, thereby executing the reaction simulation. When the execution of the reaction simulation completes, the control unit 51 stores the execution result (yield) of the reaction simulation in the storage unit 59 in association with the reactive condition.

The ranking unit 572 assigns the rank (performs ranking) to each of the plurality of reactive conditions associated with the yields and stored in the storage unit 59 according to a predetermined condition. For example, the ranking is performed with a case where the yield is 80% or more as “A”, a case where the yield is less than 80% and 50% or more as “B”, and a case where the yield is less than 50% as “C”. The determination unit 573 determines whether the reactive condition under which the yield meets the predetermined condition is present or not. Specifically, whether the reactive condition with a predetermined rank or more is present or not is determined (for example, whether the reactive condition with the rank “B” or more is present or not is determined) based on the rank assigned by the ranking unit 572.

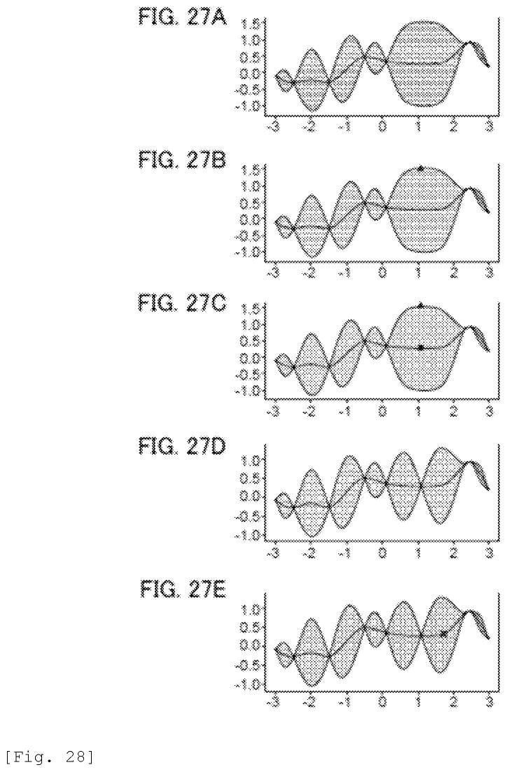

The second reactive condition selection unit 574 selects the reactive condition determined to meet the predetermined condition (reactive condition determined to have the predetermined rank or more) by the determination unit 573 as the output object, and further, assigns a display priority to the reactive condition selected as the output object. When it is determined that there is no reactive condition that meets the predetermined condition (when it is determined that there is no reactive condition with the predetermined rank or more) by the determination unit 573, the third optimization unit 575 executes the optimization process in searching (resetting) the reactive condition. Note that while the process here will be described by referring to the drawings from FIG. 27A to FIG. 29 described later, for the setting (sweep) of the reactive condition, the random search, the grid search, or the like is applicable in addition to the Bayesian optimization applied in this embodiment.

When it is determined that there is a reactive condition that meets the predetermined condition (when it is determined that there is a reactive condition with the predetermined rank or more) by the determination unit 573, the display control unit 58 controls the external display device 7 to display the reactive condition selected as the output object by the second reactive condition selection unit 574 according to the display priority. When it is determined that there is no reactive condition that meets the predetermined condition by the determination unit 573, the display control unit 58 performs the control to display the result (reactive condition) of executing the optimization process in the third optimization unit 575.

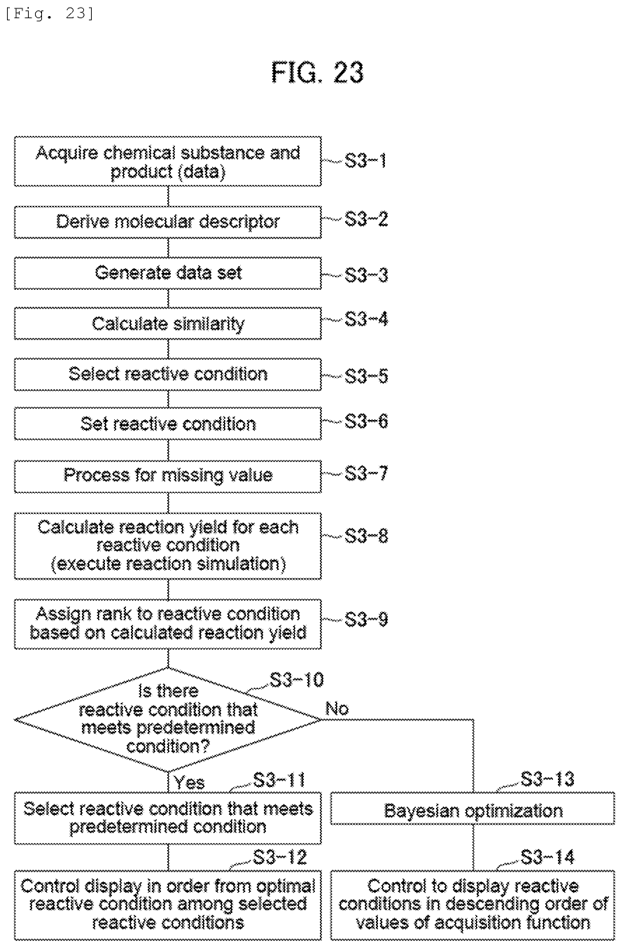

Next, with reference to the flowchart of FIG. 23, the procedure for the process of estimating (providing) the reactive condition by the reactive condition providing device 5 will be described. In S3-1, the reactive condition providing device 5 acquires the information on the plurality of chemical substances (information on the chemical substance A described with SMILES notation and information on the chemical substance B described with SMILES notation) and the information on the product (information on the product described with SMILES notation) by the data acquisition unit 52.

In S3-2, the reactive condition providing device 5 derives the molecular descriptors based on the information on the chemical substance and the information on the product by the molecular descriptor derivation unit 532. The process here is a process similar to the case of the model generation device, and the molecular descriptor derivation unit 532 executes the calculation of the chemical structure information and the acquisition of the physical property information. In S3-3, the reactive condition providing device 5 generates a predetermined data set by adding the molecular descriptor derived in S3-2 to the information on the chemical substance as a component constituting the data, and further adding the molecular descriptor to the information on the product as a component of the data set by the data set generation unit 531.

In S3-4, the reactive condition providing device 5 calculates the similarity between the information on the plurality of chemical substances acquired by the data acquisition unit 52 and the information on a plurality of chemical substances reacted in the past stored in the reactive condition database by the similarity calculation unit 542. Also for the product, similarly, the reactive condition providing device 5 calculates the similarity between the information on the product acquired by the data acquisition unit 52 and the information on a product produced as a result of a reaction in the past by the similarity calculation unit 542.

In S3-5, the reactive condition providing device 5 selects the reactive conditions by comparing the similarity calculated in S3-4 with a predetermined threshold by the first reactive condition selection unit 543. In S3-6, the reactive condition providing device 5 sets assumed combinations of the reactive conditions from the reactive conditions selected in S3-5 by the reactive condition range setting unit 544.

In S3-7, when the reactive condition set by the reactive condition range setting unit 544 does not meet a predetermined requirement as the data set, the reactive condition providing device 5 performs predetermined missing value processing to the reactive condition (data set) by the missing value processing unit 55.

In S3-8, the reactive condition providing device 5 executes the reaction simulation by inputting the reactive condition set in S3-6 (among the reactive conditions, when a reactive condition includes a missing value, one in which the predetermined missing value processing has been performed to the reactive condition) to the estimation model 56 by the reaction simulation unit 571 of the analysis unit 57. When the execution of the reaction simulation completes, the control unit 51 stores the execution result (yield) of the reaction simulation in the storage unit 59.

In S3-9, the reactive condition providing device 5 assigns the rank (performs ranking) to each of the plurality of reactive conditions stored in the storage unit 59 based on the yields (execution result of the reaction simulation) associated with the reactive conditions by the ranking unit 572 of the analysis unit 57. In S3-10, the reactive condition providing device 5 determines whether the reactive condition with the predetermined rank or more is present or not based on the rank assigned in S3-9 by the determination unit 573 of the analysis unit 57. When it is determined that a reactive condition with the predetermined rank or more is present in the plurality of reactive conditions (S3-10 Yes) by the determination unit 573 of the analysis unit 57, the reactive condition providing device 5 transitions the process to S3-11. When it is determined that a reactive condition with the predetermined rank or more is not present in the plurality of reactive conditions (S3-10 No), the process is transitioned to S3-13.

When it is determined that the reactive condition with the predetermined rank or more is present, in S3-11, the reactive condition providing device 5 selects the reactive condition determined to meet the predetermined condition (reactive condition determined to have the predetermined rank or more) in S3-10 as an output object, and further, assigns a display priority to the reactive condition selected as the output object by the second reactive condition selection unit 574 of the analysis unit 57. In S3-12, the reactive condition providing device 5 controls the external display device 7 to display the reactive condition selected as the output object by the second reactive condition selection unit 574 in S3-11 according to the display priority by the display control unit 58.

When it is determined that the reactive condition with the predetermined rank or more is not present, in S3-13, the reactive condition providing device 5 optimizes (resets) the reactive condition by the third optimization unit 575. Note that while the process here will be described later with reference to the drawings from FIG. 27A to FIG. 29, when the number of yields (experimental points) obtained by the experiment is insufficient in the execution of the optimization, the experimental point can be virtually set based on the result of executing the simulation using the estimation model. In S3-14, the reactive condition providing device 5 performs a control of displaying the reactive conditions obtained as the result of executing the optimization process in S3-13 in descending order of the values of acquisition functions.

Next, with reference to FIG. 24A to FIG. 24E, the process from S3-4 to S3-6 in the flowchart (FIG. 23) illustrating the procedure for the above-described process of estimating (providing) the reactive condition by the reactive condition providing device 5 will be specifically described. That is, the process of calculating the similarity (S3-4), the process of selecting the reactive condition (S3-5), and the process of selecting the reactive condition (S3-6) will be described by referring to FIG. 24A to FIG. 24E (particularly, FIG. 24C to FIG. 24E).

FIG. 24A illustrates the information on a plurality of chemical substances (information on the chemical substance A and the chemical substance B) and the information on a product, which are acquired by the data acquisition unit 52 (left side of FIG. 24A), and the information on a plurality of chemical substances (chemical substance 1 to chemical substance 8) reacted in the experiments performed in the past, the information on products (product 1 to product 4) produced as the result of reacting the plurality of chemical substances, and the information on the reactive conditions when the plurality of chemical substances are reacted, which are stored in the reactive condition database 541 (right side of FIG. 24A). In FIG. 24A, the information on a plurality of chemical substances and the information on products are described with SMILES notation. The information on the reactive conditions when the plurality of chemical substances are reacted is indicated as reaction items.

FIG. 24B illustrates the information on a plurality of chemical substances (information on the chemical substance A and the chemical substance B) and the information on a product (left side of FIG. 24A), which are illustrated in the left side of FIG. 24A, converted into the chemical structure information (fingerprints) by the molecular descriptor derivation unit 532 (left side of FIG. 24B). FIG. 24B illustrates the information on a plurality of chemical substances (chemical substance 1 to chemical substance 8) and the information on products (product 1 to product 4) produced as the result of reacting the plurality of chemical substances converted into the chemical structure information (fingerprints) by the molecular descriptor derivation unit 532, and the information on the reactive conditions when the plurality of chemical substances are reacted, which are illustrated in the right side of FIG. 24A (right side of FIG. 24B).

FIG. 24C illustrates the calculation result of the similarity between the information on a plurality of chemical substances (chemical structure information of the chemical substance A and the chemical substance B) in the left side of FIG. 24B and the information on a plurality of chemical substances (chemical structure information of the chemical substance 1 to the chemical substance 8) in the right side of FIG. 24B, and the similarity between the information on a product (chemical structure information of the product) in the left side of FIG. 24B and the information on a plurality of products (chemical structure information of the product 1 to the product 4) in the right side of FIG. 24B using the above-described Tanimoto coefficient. The calculated similarities are associated the information on the reactive conditions.

For example, in FIG. 24C, the uppermost row illustrates, in the order from the left, (1) the similarity between the chemical structure information of the chemical substance A acquired by the data acquisition unit 52 and the chemical structure information of the chemical substance 1 reacted in the experiment performed in the past stored in the reactive condition database 541 and the calculation formula (Tanimoto coefficient), (2) the similarity between the chemical structure information of the chemical substance B acquired by the data acquisition unit 52 and the chemical structure information of the chemical substance 2 reacted in the experiment performed in the past stored in the reactive condition database 541 and the calculation formula (Tanimoto coefficient), (3) the similarity between the chemical structure information of the product acquired by the data acquisition unit 52 and the chemical structure information of the product 1 produced as the result of reacting the chemical substance 1 with the chemical substance 2 stored in the reactive condition database 541 and the calculation formula (Tanimoto coefficient), and (4) the reactive conditions (temperature, concentration, and solvent) when the chemical substance 1 is reacted with the chemical substance 2.

FIG. 24D illustrates the reactive conditions selected by comparing the similarities calculated in FIG. 24C with predetermined thresholds. Specifically, a mean value of the similarity between the chemical structure information of the chemical substance A acquired by the data acquisition unit 52 and the chemical structure information of the chemical substance reacted in the experiment performed in the past stored in the reactive condition database 541, the similarity between the chemical structure information of the chemical substance B acquired by the data acquisition unit 52 and the chemical structure information of the chemical substance reacted in the experiment performed in the past stored in the reactive condition database 541, and the similarity between the chemical structure information of the product acquired by the data acquisition unit 52 and the chemical structure information of the product produced as the result of reacting the chemical substances stored in the reactive condition database 541 is calculated, and the reactive conditions selected by comparing the mean value with the predetermined threshold are illustrated. Here, the predetermined threshold is set to 0.8, and the reactive conditions with the mean value of the similarity of 0.8 or more are selected and illustrated.

In FIG. 24D, as described above, the reactive conditions are selected using the mean of the similarity between the chemical structure information of the chemical substance A acquired by the data acquisition unit 52 and the chemical structure information of the chemical substance reacted in the experiment performed in the past stored in the reactive condition database 541, the similarity between the chemical structure information of the chemical substance B acquired by the data acquisition unit 52 and the chemical structure information of the chemical substance reacted in the experiment performed in the past stored in the reactive condition database 541, and the similarity between the chemical structure information of the product acquired by the data acquisition unit 52 and the chemical structure information of the product produced as the result of reacting the chemical substances stored in the reactive condition database 541. However, a weighted average may be used in selecting the reactive condition. For example, the mean may be used with the relatively highly set weight of the similarity between the chemical structure information of the product acquired by the data acquisition unit 52 and the chemical structure information of the product produced as the result of reacting the chemical substances stored in the reactive condition database 541.

FIG. 24E illustrates the combinations of the reactive conditions assumed from the reactive conditions selected in FIG. 24D. Here, as the reactive condition, as illustrated in FIG. 24D, the temperature, the concentration, and the solvent constitute one set, and on the premise that there are two sets (that is, on the premise that the reactive conditions of the temperature, the concentration, and the solvent each have two values), eight (=2×2×2) combinations are generated, and the combinations are illustrated. As illustrated in FIG. 24A to FIG. 24E, by thus executing the data processing, the reactive condition is set.

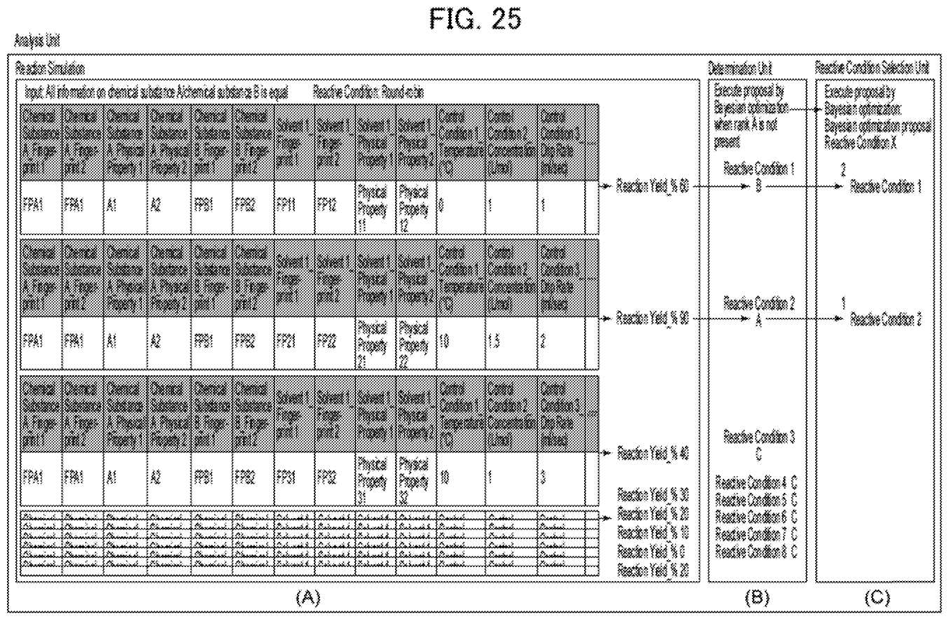

Then, when the reactive condition is thus set, the reaction simulation is executed by inputting the set reactive condition (when the set reactive condition includes a missing value, a reactive condition to which the missing value processing has been performed) to the estimation model 56. Next, with reference to FIG. 25A to FIG. 25C, the process from S3-8 to S3-12 in the flowchart (FIG. 23) illustrating the procedure for the above-described process of estimating (providing) the reactive condition by the reactive condition providing device 5 will be specifically described. That is, the process of executing the reaction simulation (S3-8), the process of ranking (S3-9), the process of determining whether the reactive condition that meets the predetermined condition is present or not (S3-10), and the process of selecting the reactive condition that meets the predetermined condition (S3-11) will be described.

FIG. 25A illustrates a data set in which the above-described reactive conditions set as illustrated in FIG. 24 are added to (merged with) the predetermined data set generated in S3-3 (that is, data set including the molecular descriptor and the physical property information as the components). By inputting the data set of FIG. 25A to the estimation model 56, the reaction simulation is executed.

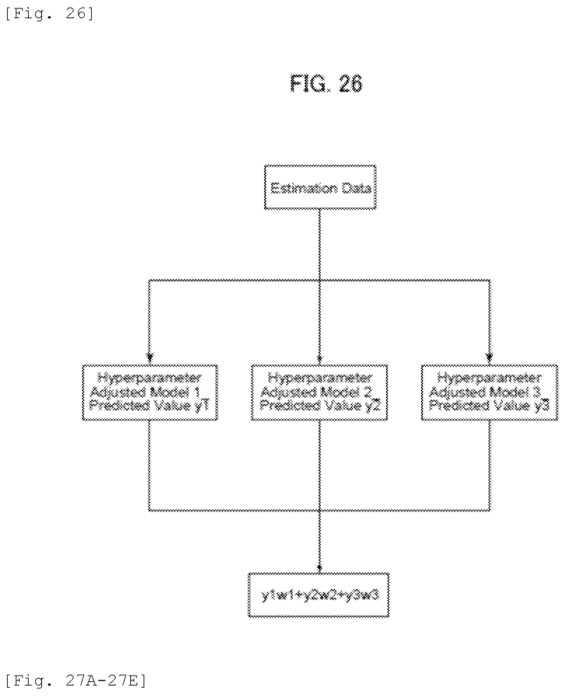

The executing process of the reaction simulation here can be described in detail with reference to FIG. 26. FIG. 26 is a drawing illustrating the reaction simulation process when a plurality of estimation models (three estimation models) is used. As illustrated in FIG. 26, the data set of FIG. 25A is input to each of the three estimation models.

Each of the estimation models executes the calculation process to the input (data set of FIG. 25A), and calculates a predicted value. In FIG. 26, from the left side of FIG. 26, the estimation model 1 calculates the predicted value as y1, the estimation model 2 calculates the predicted value as y2, and the estimation model 3 calculates the predicted value as y3. Then, when the predicted values are calculated in the respective estimation models, the calculated predicted values are multiplied by the weights set to the respective estimation models, and further, the process of adding up them is executed.

That is, the process of multiplying the calculated predicted value y1 by the weight w1 set to the estimation model 1, multiplying the calculated predicted value y2 by the weight w2 set to the estimation model 2, multiplying the calculated predicted value y3 by the weight w3 set to the estimation model 3, and adding up the multiplying results is executed. Then, the result of adding up (that is, the result of calculating y1×w1+y2×w2+y3×w3) is output as the yield.

With reference to FIG. 25A again, on the right side in the data set of FIG. 25A, the calculated yields are indicated.

FIG. 25B illustrates the ranks assigned to the calculated yields. Here, the ranks are assigned with the case where the yield is 80% or more as “A,” the case where the yield is less than 80% and 50% or more as “B,” and the case where the yield is less than 50% as “C.”

In FIG. 25B, the reactive condition 2 is ranked as “A,” the reactive condition 1 is ranked as “B,” and the others (reactive conditions 3 to 8) are ranked as “C.” Based on this premise, the determination unit 573 of the analysis unit 57 determines whether reactive the condition with the predetermined rank of “B” or more is present or not. Here, since the reactive condition 2 is “A” and the reactive condition 1 is “B,” it is determined that the reactive condition with the rank “B” or more is present. Note that when the reactive condition with the predetermined rank or more is not present (that is, here, when the reactive condition with the rank “B” or more is not present), as described as the transition from S3-10 to S3-13 in the above described flowchart of FIG. 23, the third optimization unit 575 of the analysis unit 57 executes the optimization process, thereby searching the reactive condition.

FIG. 25C illustrates that the reactive condition 1 and the reactive condition 2 are selected as the reactive conditions with the predetermined rank or more, and further, the display priorities are set corresponding to the ranks (when the ranks are the same, yields). Here, since the rank of the reactive condition 2 is “A” and the rank of the reactive condition 1 is “B” in the selected reactive conditions, the display priority of the reactive condition 2 is set to 1 and the display priority of the reactive condition 1 is set to 2 (that is, setting is made such that the reactive condition 2 is displayed prior to the reactive condition 1).

Next, with reference to FIG. 27A to FIG. 29, the process of the Bayesian optimization executed when the reactive condition output as the estimation result does not meet the predetermined condition (yield) will be described. As described above, the reactive condition providing device 5 executes the Bayesian optimization when the reactive condition output as the estimation result does not meet the predetermined condition (yield) (in the case of S3-10 No in the flowchart of FIG. 23), and outputs the reactive conditions based on the execution result of the Bayesian optimization. The Bayesian optimization is provided as a method for obtaining the maximum value of the yield (here, a method for obtaining the reactive condition under which the yield has the maximum value) when the yield is determined as a function of the reactive condition.

In this embodiment, (instead of executing the Bayesian optimization once and outputting (providing) one reactive condition), the Bayesian optimization is executed multiple times at once, and a plurality of the reactive conditions are provided. Thus, by providing a plurality of the reactive conditions to an operator at once, the operator can concurrently perform experiments (operations) in some cases, and consequently, the man-hour for operation can be reduced. Note that when the Bayesian optimization is executed multiple times at once, Constant Liar Approach is applied.

The following describes the contents of the process here as a supplementary explanation with reference to FIG. 27A to FIG. 27E. For convenience of explanation, a case where the number of the reactive conditions as the feature quantities is one (for example, temperature) will be described. FIG. 27A illustrates a function (prediction line) predicted based on eight experimental points (results) having the horizontal axis indicating the temperature (reactive condition) and the vertical axis indicating the yield, and also illustrates the variance in a veil shape. The eight experimental points are set based on the yields actually obtained by the experiment. Note that when the number of the yields obtained by the experiment is insufficient for executing the Bayesian optimization, it is also possible to set the experimental points based on the estimation result using the estimation model (S3-8 in the flowchart of FIG. 23).

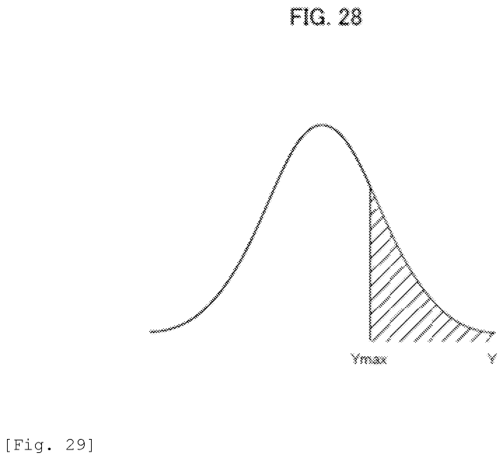

In this case, the third optimization unit 575 determines a next experimental point using a predetermined acquisition function. As the acquisition function, for example, Expected Improvement (EI), Probability of Improvement (PI), Upper Confidence Bounds (UCB), or the like can be used, and here, an example of PI will be described. PI is indicated as a value (hatched part of FIG. 28) obtained by integrating from champion data Ymax (data indicated as the highest yield among the yields obtained so far) to the infinity in the normal distribution including the sum of the mean value and the variance value as the component, and a temperature at which the integrated value becomes highest is selected (identified) as a temperature with a high possibility of exceeding the yield of the champion data. Note that the normal distribution here is a kind of probability distribution, and expressed by a formula below.

$$\frac{1}{\sqrt{2 π {σ}^{2}}} exp ( - \frac{{( x - μ )}^{2}}{2 {σ}^{2}} ) \qquad [ Math . 2 ]$$

The value obtained by integrating from the champion data Ymax to the infinity can be calculated by a formula below. In the formula below, ε is used corresponding to the degree of variance in a manner in which a point considerably close to the champion data Ymax is not selected as the next experimental point, and for example, set to 0.01 or the like.

$$PI ( {x}^{( n + 1 )} ) = {\int }_{Ymax + ε}^{∞} \frac{1}{\sqrt{2 π {σ}^{2} ( {x}^{( n + 1 )} )}} exp \{ - \frac{1}{2 {σ}^{2} ( {x}^{( n + 1 )} )} {( x - μ ( {x}^{( n + 1 )} ) )}^{2} \} dx \qquad [ Math . 3 ]$$

Then, by calculating the above formula while shifting the reactive condition (temperature), x⁽ⁿ⁺¹⁾ at which the value of PI(x⁽ⁿ⁺¹⁾) becomes highest is identified (that is, x⁽ⁿ⁺¹⁾ at which the area of the hatched part of FIG. 28 becomes largest (that is, next sample) is identified). In FIG. 27B, the temperature indicated by a triangle is identified as a temperature with a high possibility of exceeding the yield of the champion data in the acquisition function.

In this embodiment, instead of immediately performing the experiment (operation) to measure the yield based on the provided temperature, as described above, Constant Liar Approach is applied and the Bayesian optimization is further executed assuming that the experimental point lies on the prediction line corresponding to the provided temperature (assuming to be a position of square of FIG. 27C).

When the Bayesian optimization is further executed, the relation (function) between the temperature and the yield is illustrated as FIG. 27D, and the temperature with a high possibility of exceeding the yield of the champion data is identified to the function using the acquisition function (FIG. 27E). Furthermore, assuming that the experimental point lies on the prediction line corresponding to the provided temperature (assuming to be a position of x in the drawing), the Bayesian optimization is further executed.

Thus, by executing the Bayesian optimization multiple times at once, a plurality of the reactive conditions is output (provided). That is, the reactive conditions are output at once by the number of the experiments (operations) that the operator can concurrently perform. The following describes the procedure for the process here using the flowchart as a supplementary explanation.

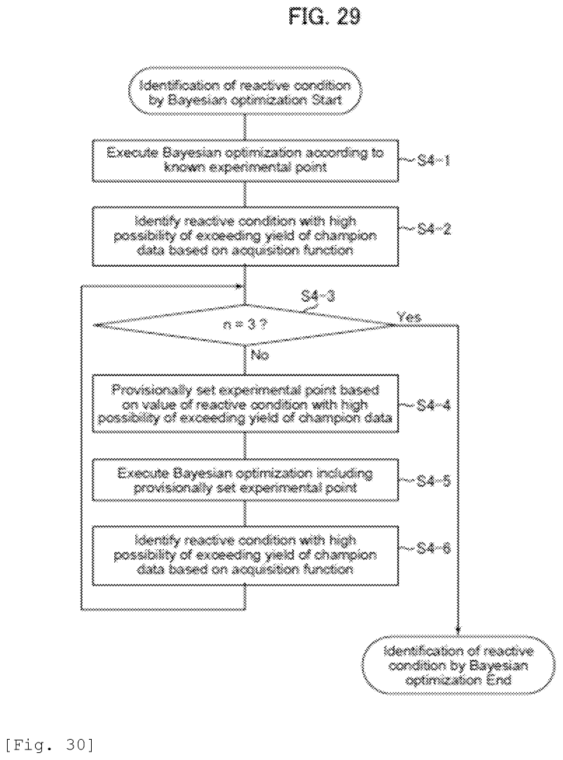

FIG. 29 is a flowchart illustrating the procedure for the process of executing the Bayesian optimization with the application of Constant Liar Approach to identify the reactive condition. In the flowchart of FIG. 29, the start of the process is, for example, triggered by that the reactive condition output as the estimation result does not meet the predetermined condition (yield).

In S4-1, the third optimization unit 575 executes the Bayesian optimization according to known experimental points. As described above, when the number of the known experimental points is insufficient for executing the Bayesian optimization, experimental points are set based on the result of the estimation using the estimation model. In S4-2, the third optimization unit 575 identifies the reactive condition with a high possibility of exceeding the yield of the champion data based on the acquisition function.

In S4-3, the third optimization unit 575 determines whether the number of the identified reactive conditions reaches a predetermined number or not. Here, a variable nis set to 1 as an initial value (n=1 is set), and when three reactive conditions are identified (set), the process of identifying the reactive condition ends. The predetermined number can be set by the operator corresponding to the experience of the operator and the contents of the experiment. As the predetermined number, in addition to the number of the experiments that the operator can concurrently perform, for example, the number of experiment candidates when it is wanted to know another experiment candidate as a reference, or the number of concurrent outputs when it is wanted to keep the feature quantity in a specific range (for example, it is wanted to keep the temperature to 50° C. or less) can be examined.

In S4-4, as described above with reference to FIG. 27, the third optimization unit 575 provisionally sets the experimental point based on the value of the reactive condition with a high possibility of exceeding the yield of the champion data. In S4-5, the third optimization unit 575 executes the Bayesian optimization again including the provisionally set experimental point. In S4-6, the third optimization unit 575 identifies the reactive condition with a high possibility of exceeding the yield of the champion data based on the acquisition function.

Then, when the process of S4-6 is executed, the control unit 51 counts up the variable n (not illustrated in the flowchart of FIG. 29) (that is, 1 is added to the variable n to set n to 2), and returns the process to S4-3. Thus, the process from S4-3 to S4-6 is repeatedly executed until the variable n reaches a predetermined number, and when the variable n reaches the predetermined number, the process illustrated in the flowchart of FIG. 29 ends. By thus executing the process, the reactive conditions can be output by the number of the operations that can be concurrently performed, and as described above, the man-hour for operation can be reduced.

While the number of the reactive conditions (that is, feature quantities) is one in the above-described explanation and the description is given two-dimensionally in FIG. 27A to FIG. 27E, actually, a plurality of the reactive conditions are set, and therefore, the calculation is executed by computing a matrix or the like in the reactive condition providing device. Although not illustrated in FIG. 22, the third optimization unit 575 includes, as the functions, a derivation unit that derives the reactive condition under which the yield meets the predetermined condition using the predetermined acquisition function (for example, the reactive condition under which the yield is the highest, the reactive condition under which the yield is equal to or more than the predetermined threshold, or the like), and a setting unit that provisionally sets the experimental point from the reactive condition derived by the derivation unit using the function of the yield to the reactive condition.

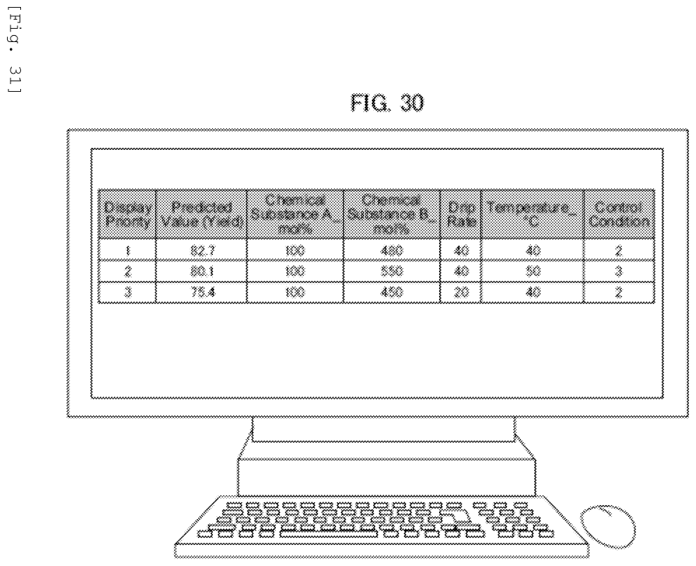

FIG. 30 is a drawing illustrating a sample screen of the reactive conditions displayed on the external display device 7. FIG. 30 illustrates that the three reactive conditions selected as the reactive conditions with the predetermined rank or more by the second reactive condition selection unit 574 of the analysis unit 57 are displayed according to the assigned display priorities.

As illustrated in FIG. 30, the displayed reactive conditions (drip rate, temperature, control condition) are associated with the display priority, the predicted value (yield) when the chemical substance A is reacted with the chemical substance B, and the chemical substance A and the chemical substance B as reaction object materials to constitute one data set, and the data sets are displayed in the descending order according to the display priority.

The operator selects the reactive condition displayed on the display device 7 based on the easy availability of materials, the cost, and the law, and performs the experiment (operation) according to the reactive condition. Note that by inputting the experimental result to the model generation device 1 and further executing the machine learning, a model capable of providing the reactive condition with higher accuracy can be generated.

Additionally, as a supplementary explanation, while the explanation is given using the information on two chemical substances, the information on the chemical substance A and the information on the chemical substance B, as the information on a plurality of chemical substances in the above-described embodiment, the reactive condition can be provided even when information on three or more chemical substances is input. When the information on three or more chemical substances is input, as the components constituting the chemical property information generated as the predetermined data set, for example, the chemical structure information and the physical property information of chemical substances such as a chemical substance C and a chemical substance D in addition to the chemical substance A and the chemical substance B are added, and it is only necessary to execute the similarity calculation process, the process of selecting and setting the reactive condition, and the reaction simulation on the premise of the data set (chemical property information) configured thus. In this case, it is only necessary to store the experimental data of reacting three or more chemical substances in the reactive condition database. The same applies to the Bayesian optimization of S3-13.

As described above, according to the embodiment, under the condition that the acquired information on the plurality of chemical substances and the product is similar to the information on the plurality of chemical substances and the product set to the reactive condition (or the condition of being the same), a plurality of the acquired reactive conditions when a plurality of chemical substances is reacted are set from the reaction items set to the reactive conditions. Furthermore, using the estimation model in which the machine learning has been executed with the chemical structure information and the physical property information of a plurality of chemical substances reacted in the past, the reactive conditions in the reaction, and the yields when reactions are performed under the reactive conditions as the training data, the yield is estimated for each of the plurality of reactive conditions, and the reactive conditions under which the yields among the estimated yields meet the predetermined condition are displayed (output). Accordingly, in the setting of the reactive condition under which the yield meets the predetermined condition, the man-hour for operation can be reduced.

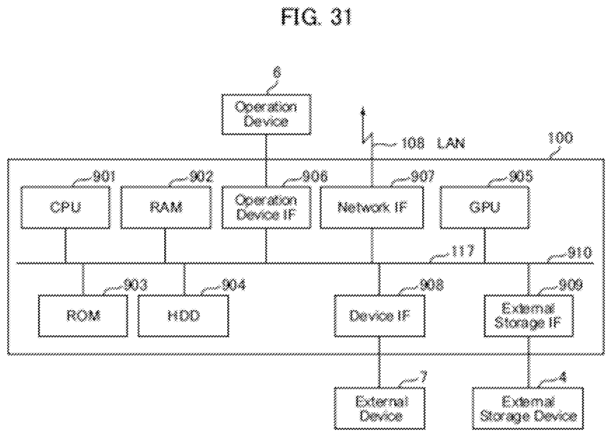

FIG. 31 is a block diagram illustrating a hardware configuration of the model generation device 1. As illustrated in FIG. 31, the model generation device 1 includes a CPU 901, a RAM 902, a ROM 903, a HDD 904, a GPU 905, an operation device interface 906, a network interface 907, a device interface 908, an external storage interface 909. The model generation device 1 is connected to an external device (display device 7) and the external storage device 4 in addition to the operation device connected via the operation device interface 906.

The CPU (Central Processing Unit) 901 is a processing unit that integrally controls each of the blocks of the model generation device 1. The CPU 901 is connected to the RAM 902, the ROM 903, the HDD 904, the GPU 905, the operation device interface 906, the network interface 907, the device interface 908, and the external storage interface 909 via a system bus 910.

The RAM (Random Access Memory) 902 includes a storage area that temporarily stores the arithmetic processing result of the CPU 901, various setting values, parameters, and the like, and a load area of various control programs. The ROM (Read Only Memory) 903 stores various programs (for example, a boot program). The HDD (Hard Disk Drive) 904 stores the physical property information, the reactive conditions, the generated estimation models, and the like. The functions of the model generation device are achieved by, for example, reading the program stored in the ROM 903 to the RAM 902 and executing the program by the CPU 901.

The GPU (Graphics Processing Unit) 905 is a processing unit for executing the computing of the process at a high speed, and executes a predetermined calculation (for example, a matrix operation executed in the generation stage of the estimation model, or the like) transferred from the CPU 901. The operation device interface 906 is an interface for inputting (acquiring) data to/from the operation device 6. The network interface 907 is connected to a Local Area Network (LAN) wired or wirelessly, and allows input and output of information with external devices (for example, allows updating the physical property information, the reactive conditions, and the various programs). The device interface 908 is an interface for the connection to, for example, an external analysis device or the like. The external storage interface 909 is an interface for the connection to an external storage device. For example, when the model generation device and the reactive condition providing device are configured as different devices, the external storage interface 909 is used for storing the estimation model generated by the model generation device 1 and stored in the HDD in an external storage device via the external storage interface 909.

Note that the hardware configuration of the reactive condition providing device 5 is almost similar to the hardware configuration of the model generation device 1, but various storage elements, objects (data) stored in the storage device, and external devices (for example, display device 7 or the like) connected via the device interface 908 are different.

Additionally, the present invention can be achieved by a process of providing a program that achieves one or more functions of the above-described embodiments to a device via a network or a storage medium, and reading and executing the program by one or more processors in a computer of the device.

While a part of the whole of the above-described embodiment can be described as an additional remark below, the above-described embodiment is not limited thereto.

### Additional Remark

A reactive condition providing device that includes:
optimization unit that optimizes a function of a yield to a reactive condition based on an experimental point in searching the reactive condition;
deriving unit that derives a reactive condition under which the yield meets a predetermined condition using a predetermined acquisition function;
setting unit that provisionally sets an experimental point from the derived reactive condition using the function of the yield to the reactive condition; and
display control unit that causes a display device to display the reactive condition derived by the deriving unit, wherein
the optimization of the function of the yield to the reactive condition, the deriving of the reactive condition under which the yield meets the predetermined condition, and the setting of the experimental point are repeatedly executed by a predetermined number of times.

### EXPLANATION OF LETTERS OR NUMERALS

1 model generation device
2 analysis device
3 reactor
4 external storage device
5 providing device
6 operation device
7 display device
11 control unit
12 data acquisition unit
13 training data generation unit
14 preprocessing unit
15 feature quantity selection unit
16 estimation model generation unit
17 storage unit
51 control unit
52 data acquisition unit
53 chemical property information generation unit
54 reactive condition setting unit
55 missing value processing unit
56 estimation model
57 analysis unit
58 display control unit
59 storage unit

1. An estimation model generation method comprising:
an acquiring step of acquiring information on a plurality of chemical substances reacted in a past, reactive conditions in the reactions, information on a product produced by reacting the plurality of chemical substances, and yields when the reactions are performed under the reactive conditions;
a first generating step of generating training data from chemical structure information and physical property information derived for each piece of the information on the plurality of chemical substances reacted in the past, the reactive conditions, chemical structure information and physical property information derived for the information on the product, and the yields; and
a second generating step of generating an estimation model having information on a plurality of chemical substances to be reacted and information on a product to be produced as input values and a reactive condition under which a yield of the product to be produced meets a predetermined condition as an output value by executing machine learning using the training data.

2. The estimation model generation method according to claim 1, wherein the second generating step
generates the estimation model based on feature quantities as components constituting the training data, and
includes:
a first storing step of storing verified accuracies of the generated estimation models in association with the estimation models;
a calculating step of calculating importance degrees of the feature quantities in the generated estimation models;
a determining step of determining whether the feature quantities include a deletable feature quantity or not based on properties of the feature quantities and the importance degrees of the feature quantities; and
a deleting step of deleting the feature quantity determined to be deletable from the components constituting the training data when the deletable feature quantity is determined to be present in the determining step, wherein
the generation of the estimation models, the first storing step, the calculating step, the determining step, and the deleting step are repeatedly executed in sequence until it is determined that no deletable feature quantity is included in the feature quantities in the determining step, and an estimation model in which the verified accuracy meets a predetermined condition is selected among the estimation models stored in the first storing step.

3. The estimation model generation method according to claim 1, wherein
the second generating step includes:
an optimizing step of optimizing a hyperparameter set to an algorithm of the machine learning; and
a second storing step of generating the estimation models with the optimized hyperparameter and storing verified accuracies of the generated estimation models in association with the estimation models, wherein
the optimizing step, the generation of the estimation models, and the second storing step are repeatedly executed in sequence until an expected improvement degree output in the optimizing step meets s a predetermined condition, and when the expected improvement degree meets the predetermined condition, an estimation model in which the verified accuracy meets the predetermined condition among the estimation models stored in the second storing step is selected.

4. The estimation model generation method according to claim 1, further comprising:
a selecting step of selecting a feature quantity used for generating the estimation model;
a dividing step of dividing the training data corresponding to a count of a plurality of estimation models to be further generated;
a third generating step of generating a plurality of estimation models by executing machine learning using a predetermined machine learning method based on the selected feature quantity for each of the divided training data; and
a setting step of setting a predetermined weight for each of the plurality of generated estimation models.

5. The estimation model generation method according to claim 4, wherein
in the dividing step, the training data is divided based on a bootstrap method.

6. The estimation model generation method according to claim 4, wherein
the predetermined machine learning method is any of a decision tree, a neural network, a gradient boosting, and a support vector, and
a part of estimation models among the plurality of generated estimation models is allowed to be generated by executing the machine learning using an identical machine learning method.

7. The estimation model generation method according to claim 4, wherein
the predetermined weights set to the plurality of generated estimation models are estimated by Bayesian optimization.

8. The estimation model generation method according to claim 1, further comprising
a step of performing a predetermined preprocessing corresponding to the training data generated in the first generating step when the training data does not meet a predetermined requirement.

9. The estimation model generation method according to claim 8, wherein
the preprocessing is a processing of at least any of missing value processing, a category value conversion process, or a data standardization or normalization.

10. A non-transitory computer readable storage medium storing instructions that cause a computer to generate an estimation model, the instructions configured to cause the computer to perform functions, comprising:
acquiring information on a plurality of chemical substances reacted in a past, reactive conditions in the reactions, information on a product produced by reacting the plurality of chemical substances, and yields when the reactions are performed under the reactive conditions;
generating training data from chemical structure information and physical property information derived for each piece of the information on the plurality of chemical substances reacted in the past, the reactive conditions, chemical structure information and physical property information derived for the information on the product, and the yields; and
generating an estimation model having information on a plurality of chemical substances to be reacted and information on a product to be produced as input values and a reactive condition under which a yield of the product to be produced meets a predetermined condition as an output value by executing machine learning using the training data.

11. An estimation model generation device comprising:
a processor programmed to:
acquire information on a plurality of chemical substances reacted in a past, reactive conditions in the reactions, information on a product produced by reacting the plurality of chemical substances, and yields when the reactions are performed under the reactive conditions;
generate training data from chemical structure information and physical property information derived for each piece of the information on the plurality of chemical substances reacted in the past, the reactive conditions, chemical structure information and physical property information derived for the information on the product, and the yields; and
generate an estimation model having information on a plurality of chemical substances to be reacted and information on a product to be produced as input values and a reactive condition under which a yield of the product to be produced meets a predetermined condition as an output value by executing machine learning using the training data.

12. A reactive condition providing device comprising:
a memory that stores one or more reactive conditions; and
a processor programmed to:
acquire information on a plurality of chemical substances as reaction objects and information on a product to be produced;
set a plurality of reactive conditions for reacting the plurality of chemical substances as the reaction objects from reaction items set to the one or more reactive conditions when a similarity between the information on the plurality of chemical substances as the reaction objects and the information on the product to be produced and information on a plurality of chemical substances and information on a product set to the reactive conditions is equal to or more than a predetermined threshold;
estimate a yield for each of the plurality of reactive conditions from chemical structure information and physical property information derived for each piece of the information on the plurality of chemical substances as the reaction objects, chemical structure information and physical property information derived for the information on the product to be produced, and the plurality of set reactive conditions, by using an estimation model in which machine learning has been executed with chemical structure information and physical property information derived for each piece of information on a plurality of chemical substances reacted in a past, reactive conditions in the reactions, chemical structure information and physical property information derived for information on products produced in the reactions of the plurality of chemical substances, and yields when the reactions are performed under the reactive conditions as training data; and
cause a display device to display the reactive condition under which the yield among the yields estimated for the respective reactive conditions meets a predetermined condition.

13. The reactive condition providing device according to claim 12, wherein the processor is programmed to calculate the similarity between the information on the plurality of chemical substances as the reaction objects and the information on the product to be produced and the information on a plurality of chemical substances and the information on the product set to the one or more reactive conditions; and
calculate a mean value or a weighted average value of a similarity between the chemical structure information and the physical property information derived for each piece of the information on the plurality of chemical substances as the reaction objects and chemical structure information and physical property information derived for each piece of the information on the plurality of chemical substances set to the one or more reactive conditions and a similarity between the chemical structure information and the physical property information derived for the information on the product to be produced and chemical structure information and physical property information derived for the information on the product set to the one or more reactive conditions.

14. The reactive condition providing device according to claim 12, wherein the processor is programmed to:
determine whether the reactive condition under which the yield among the yields estimated for the respective reactive conditions meets a predetermined condition is present or not; and
assign display priorities to a plurality of reactive conditions when it is determined that a plurality of reactive conditions under which the yields meet the predetermined condition are present.

15. The reactive condition providing device according to claim 14, wherein the processor is programmed to optimize a function of the yield to the reactive condition in searching the reactive condition when it is determined that no reactive condition exists under which the yield meets the predetermined condition.

16. The reactive condition providing device according to claim 15, the processor is programmed to optimize the function of the yield to the reactive condition based on an experimental point;
derive the reactive condition under which the yield meets the predetermined condition using a predetermined acquisition function;
set the experimental point from the derived reactive condition using the function of the yield to the reactive condition;
repeatedly execute the optimization of the function of the yield to the reactive condition, the deriving of the reactive condition under which the yield meets the predetermined condition, and the setting of the experimental point are a predetermined number of times; and
cause the display to display the derived reactive condition.

17. A reactive condition providing method comprising:
an acquiring step of acquiring information on a plurality of chemical substances as reaction objects and information on a product to be produced;
a setting step of setting a plurality of reactive conditions for reacting the plurality of chemical substances as the reaction objects from reaction items set to one or more reactive conditions stored in a storage device when a similarity between the information on the plurality of chemical substances as the reaction objects and the information on the product to be produced and information on a plurality of chemical substances and information on a product set to the reactive conditions is equal to or more than a predetermined threshold;
an estimating step of estimating a yield for each of the plurality of reactive conditions from chemical structure information and physical property information derived for each piece of the information on the plurality of chemical substances as the reaction objects, chemical structure information and physical property information derived for the information on the product to be produced, and the plurality of set reactive conditions, by using an estimation model in which machine learning has been executed with chemical structure information and physical property information derived for each piece of information on a plurality of chemical substances reacted in a past, reactive conditions in the reactions, chemical structure information and physical property information derived for information on products produced in the reactions of the plurality of chemical substances, and yields when the reactions are performed under the reactive conditions as training data; and
a display control step of causing a display device to display the reactive condition under which the yield among the yields estimated for the respective reactive conditions in the estimating step meets a predetermined condition.

18. A non-transitory computer readable storage medium storing instructions that cause a computer to provide a reactive condition, the instructions configured to cause the computer to perform functions, comprising:
acquiring information on a plurality of chemical substances as reaction objects and information on a product to be produced;
setting a plurality of reactive conditions for reacting the plurality of chemical substances as the reaction objects from reaction items set to one or more reactive conditions stored in a storage device when a similarity between the information on the plurality of chemical substances as the reaction objects and the information on the product to be produced and information on a plurality of chemical substances and information on a product set to the reactive conditions is equal to or more than a predetermined threshold;
estimating a yield for each of the plurality of reactive conditions from chemical structure information and physical property information derived for each piece of the information on the plurality of chemical substances as the reaction objects, chemical structure information and physical property information derived for the information on the product to be produced, and the plurality of set reactive conditions, by using an estimation model in which machine learning has been executed with chemical structure information and physical property information derived for each piece of information on a plurality of chemical substances reacted in a past, reactive conditions in the reactions, chemical structure information and physical property information derived for information on products produced in the reactions of the plurality of chemical substances, and yields when the reactions are performed under the reactive conditions as training data; and
causing a display device to display the reactive condition under which the yield among the yields estimated for the respective reactive conditions meets a predetermined condition.
